(19) United States
(12) Patent Application Publication
(10) Pub. No.: US 2022/0145377 A1
(43) Pub. Date: May 12, 2022

(54) Device and Method for Biopolymer Identification

(71) Applicant: Universal Sequencing Technology Corporation, Canton, MA (US)

(72) Inventors: Peiming Zhang, Gilbert, AZ (US); Ming Lei, Sharon, MA (US)

(21) Appl. No.: 17/435,072

(22) PCT Filed: Feb. 28, 2020
(86) PCT No.: PCT/US2020/020504

§ 371 Date: Aug. 31, 2021

Related U.S. Application Data
(60) Provisional application No. 62/812,736, filed on Mar. 1, 2019.

(51) Int. Cl. C12Q 1/6825; C12Q 1/00; C12Q 1/6869

(52) U.S. Cl. CPC C12Q 1/6825; C12Q 1/6869; C12Q 1/005

(57) ABSTRACT

This invention provides a nanostructure device and method for the sequencing or identification of biomolecules based on in vitro template-directed enzymatic replication or synthesis.

### FIELD

Embodiments of the present invention are related to systems, methods, devices, and compositions of matter for the sequencing or identification of biopolymers using electronic signals. More specifically, the present disclosure includes embodiments which teach the construction of a system to detect biopolymers electronically based on enzymatic activities, including replication. The biopolymers in the present invention include but not limited to DNA, RNA, DNA oligos, protein, peptides, polysaccharides, etc., either natural or synthesized. The enzymes include but not limited to DNA polymerase, RNA polymerase, DNA helicase, DNA ligase, DNA exonuclease, reverse transcriptase, RNA primase, ribosome, sucrase, lactase, etc., either natural, mutated or synthesized. In the following, mainly DNA and DNA polymerase are discussed and used to illustrate the inventive concept.

### BACKGROUND OF THE INVENTION

DNA sequencing by enzymatic synthesis can be traced back to Sanger's chain termination method, by which dideoxynucleotides are selectively incorporated into DNA by DNA polymerase during in vitro replication of the target sequences.^{1,2} This enzymatic approach has been extended to next-generation sequencing (NGS) in a high throughput or real-time fashion.^{3,4} Although NGS has reduced the cost of sequencing a human genome to a range of $1000, the recent data shows that the cost reduction may have reached a bottom plateau (https://www.genome.gov/27565109/the-cost-of-sequencing-a-human-genome). One limiting factor is that NGS relies on optical signal detection, which requires a sophisticated instrument that is bulky and expensive.

The electrical readout of DNA synthesis by polymerase was stimulated by label-free detection,⁵ which has been developed as a platform used in the genome sequencing.⁶ The recent progress has shown that the electronic approach can be a hand-held device, such as the MinION sequencer (www.nanoporetech.com) that measures changes in ionic currents passing through protein nanopores for DNA sequencing, where a DNA helicase is employed to control the translocation of DNA through the nanopores.⁷ However, the protein nanopore can only achieve a low sequencing accuracy (85% with a single read⁸). Gundlach and coworkers have demonstrated that the ionic current blockage in a protein nanopore composed of Mycobacterium smegmatis porin A (known as MspA) is a collected event of four nucleotides (quadromer), and therefore there are 4⁴ (Le. 256) possible quadromers that exert a significant number of redundant current levels.^{9,10} Because the ionic current is affected by nucleotides beyond those inside the nanopore,¹¹ the notion of an atomically thin nanopore for sequencing may not be conceivable to achieve a single nucleotide resolution.

Collins and coworkers reported a single-wall carbon nanotube (SWCNT) field-effect transistor (FET) device with a Klenow fragment of DNA polymerase I tethered on it to monitor DNA synthesis.^{12,13} In the method, when a nucleotide was incorporated into a DNA strand, a brief excursion of ΔIt) below the mean baseline currents was recorded. The incorporation of different nucleotides by the enzyme results in differences in ΔI. This method can potentially be used in sequencing DNA. The carbon nanotube is a material made from just a single layer of carbon atoms locked in a hexagonal grid. Because of the rigid chemical structure, its sensing may mostly rely on electrostatic gating motions of charged side chains close to the protein attachment site. However, the carbon nanotube used in the device had a length of 0.5-1.0 μm,¹⁴ which poses a challenge to mounting a single protein molecule on it reproducibly. A prior art invention (WO 2017/024049) claims a nanoscale field-effect transistor (nanoFET) for DNA sequencing, where a DNA polymerase is immobilized with its nucleotide exit region oriented toward a carbon nanotube gate with a set of nucleotides with their polyphosphates labeled for the identification of incorporated nucleotides (FIG. 1).

Another invention (US 2017/0044605) has claimed an electronic sensor device to sequence DNA and RNA using a polymerase immobilized on a biopolymer that bridges two separate electrodes (FIG. 2). Also, a single enzyme can be directly wired to both positive and negative electrodes to complete a circuit such that all electrical currents must flow through the molecule (US 2018/0305727, WO 2018/208505). Nonetheless, the enzyme can have more than two contacting points to electrodes.

DNA has caught enormous attention in molecular electronics because of its unique base stacking structure that makes DNA a fine molecular wire for charge transfer (CT). Also, DNA's sequence and length are programmable, capable of forming error-free self-assembled nanostructures, such as DNA origami, with no need for expensive microfabrication technologies, rendering it an ideal candidate for nanoscale integrated circuits. In the last decades, programmed self-assembly of nucleic acids (DNA and RNA) has been developed for the construction of nanostructures.^{15,16} In general, a complex DNA nanostructure is assembled starting from a molecular motif, such as the Holliday junction,^{17, 18} multi-arm junction,¹⁹ double (DX) and triple crossover (TX) tiles,^{20,21} paranemic crossover (PX),²² tensegrity triangle,²³ six-helix bundle,²⁴ and single-stranded circular DNA or DNA origami (FIG. 3).²⁵ With these DNA motifs, a variety of size and shape tunable nanostructures can be readily constructed. The DNA nanostructure is less rigid than the carbon nanotube, yet more rigid than DNA duplexes or molecular wires. It can also be functionalized similarly, as does the DNA duplex. It provides a unique breadboard for the construction of an electronic biosensor with a designed structure to have desired conductivity, current fluctuations, or other electrical properties. A 10×60 nm² TX tile nanostructure was measured to have a conductance of ˜70 pS in a 45-55 nm nanogap under 90% relative humidity.²⁶ Thus, a nanogap bridged by a DNA nanostructure can be employed to construct nano-bio-devices for electronic detection or identification of molecules. It is conceivable that the conductivity of a DNA nanostructure can be tuned by its sequences, configuration, and structural dynamics. Similarly, RNA nanostructures are constructed using the RNA motifs (FIG. 4) through self-assembling.^{27,28} RNA is much more versatile in structure and function compared to DNA, and its duplex is thermodynamically more stable than the DNA counterpart. Thus, the RNA nanostructure can be an alternative to the corresponding DNA nanostructure. It has been demonstrated that RNA can mediate the electron transfer as well.²⁹

To program DNA beyond the canonical nucleobases, recently, Steven Benner and his colleagues created an eight nucleotide DNA/RNA genetic system, called Hachimoji system.⁵⁶ Besides the four naturally occurring DNA nucleotides, A, C, G, and T, they created four more unnatural DNA nucleotides, P, B, S, Z with P paired with Z (P:Z) and B paired with S (B:S), similar to G:C and A:T pairs, where P and B are purine analogs and Z and S are pyrimidine analogs with P being 2-amino-8-(1′-β-D-2′-deoxyribofuranosyl)-imidazo-[1,2a]-1,3,5-triazin-[8H]-4-one, B being 6-amino-9[(1′-β-D-2′-deoxyribofuranosyl)-4-hydroxy-5-(hydroxymethyl)-oxolan-2-yl]-1H-purin-2-one, S being 3-methyl-6-amino-5-(1′-β-D-2′-deoxyribofuranosyl)-pyrimidin-2-one, and Z being 6-amino-3-(1′-β-D-2′-deoxyribofuranosyl)-5-nitro-1H- pyridin-2-one (see FIG. 5 and reference #56). These new nucleotides can form similar double helix as the four natural nucleotides do, and also they can mix to form a DNA double helix with eight bases of GACTZPSB, which can be replicated by DNA polymerase. t In the natural DNA, G:C pairs have three hydrogen bonds but A:T pairs have only two hydrogen bonds. In contrast, both P:Z and B:S pairs have three hydrogen bonds (FIG. 5), so it can be predicted that DNA duplexes formed with those unnatural bases would be more stable and more conductive than those formed with natural DNA. The same is true for RNA made of unnatural nucleotides, where the four DNA bases PBSZ become RNA bases, P: 2-amino-8-(1′-β-D-ribofuranosyl)-imidazo-[1,2a]-1,3,5-triazin-[8H]-4-one, B: 6-amino-9[(1′-β-D-ribofuranosyl)-4-hydroxy-5-(hydroxymethyl)-oxolan-2-yl]-1H-purin-2-one, S: 2-amino-1-(1′-β-D-ribofuranosyl)-4(1 H)-pyrimidinone, and Z: 6-amino-3-(1′-β-D-ribofuranosyl)-5-nitro-1 H-pyridin-2-one, with S resembles U in natural RNA (see Reference #56).

A recent study has reported that DNA polymerase I bound to a PX motif with a Ka of ˜220 nM, and a DX motif with a K_{d} of ˜13 μM in solution.³⁰ Though, the PX motif could not function as a substrate for the polymerase extension. For DNA sequencing, ϕ29 DNA polymerase is an enzyme used in various platforms.^{9,31,32} Based on amino acid sequence similarities and its sensitivity to specific inhibitors, the ϕ29 DNA polymerase belongs to the eukaryotic-type family B of DNA-dependent DNA polymerases.³³ As any other DNA polymerase, it accomplishes sequential template-directed addition of dNMP units onto the 3′—OH group of a growing DNA chain, showing discrimination for mismatched dNMP insertion by a factor from 10⁴ to 10^{6,34} In addition, ϕ29 DNA polymerase catalyzes 3′-5′ exonucleolysis, i.e. the release of dNMP units from the 3′ end of a DNA strand, degrading preferentially a mismatched primer-terminus, which further enhances the replication fidelity.³⁵⁻³⁷ The ϕ29 DNA polymerase's proofreading activity, strand displacement, and processivity may be attributed to its unique structure (FIG. 6).³⁸⁻⁴⁰

### BRIEF DESCRIPTION OF THE DRAWINGS

FIG. 1: A prior art nanoscale field-effect transistor (nanoFET) and an exemplary set of nucleotide analogs carrying differentiable charged conductive labels for DNA sequencing.

FIG. 2: A prior art of using biopolymers to connect a DNA polymerase to electrodes.

FIG. 3: Exemplary DNA motifs for the construction of DNA nanostructures.

FIG. 4: Exemplary RNA motifs for the construction of RNA nanostructures.

FIG. 5: DFT models of Hachimoji hydrogen bonding base pairs and calculated HOMO and LUMO energies.

FIG. 6: Ribbon representation of the domain organization of ϕ29 DNA polymerase.

FIG. 7: Structures of artificial nucleobases for the construction of nucleic acid-based molecular wires.

FIG. 8: A schematic diagram of a single molecule DNA sequencing device.

FIG. 9: Kinetic mechanism of nucleotide-binding and incorporation accompanied by conformation changes of the DNA polymerase.

FIG. 10: An illustration of a process of fabricating a nanogap with a passivated substrate, passivated nanowires, and exposed silicon oxide surface in the nanogap area.

FIG. 11: Chemical structures of 5′-mercapto-nucleosides used at the end of DNA nanostructures for attachment to metal electrodes.

FIG. 12: Chemical structures of base chalcogenated nucleosides.

FIG. 13: (a) a tripod containing a carboxyl function as an anchor for attaching DNA nanostructures to metal electrodes; (b) Chemical structures of nucleosides containing an amino function at their respective nucleobases.

FIG. 14: Chemical structures of nucleobase chalcogenated nucleosides.

FIG. 15: Chemical structures of nucleobase chalcogenated nucleosides.

FIG. 16: Electrochemical functionalization of an electrode (cathode) of the nanogap using an N-heterocyclic carbene.

FIG. 17: A schematic diagram of immobilizing a DNA tile on a streptavidin in a nanogap for its attachment to electrodes.

FIG. 18: (a) Chemical structure of a four-arm linker containing two biotins and two silatrane functions; (b) its 3D structure from a molecular mechanics calculation.

FIG. 19: Chemical structures of biotinylated nucleosides.

FIG. 20: A mutant of ϕ29 DNA polymerase containing p-azidophenylalanine at the locations of 277 and 479 with two tags at its two termini as well as a mutant containing p-azidophenylalanine at the sites 277 and 479. The native structure is adopted from Protein Data Bank (PDB ID: 1XHX).³⁸

FIG. 21: A process of attaching peptides to the termini of ϕ29 DNA polymerase.

FIG. 22: A crystal structure of ϕ29 DNA polymerase complexed with primer-template DNA and incoming nucleotide substrates (PDB ID: 2PYL).

FIG. 23: Chemical structures of nucleosides containing acetylene.

FIG. 24: Chemical structures of nucleoside hexa-phosphates tagged with DNA intercalators.

FIG. 25: A schematic diagram of a single-molecule device for direct RNA sequencing.

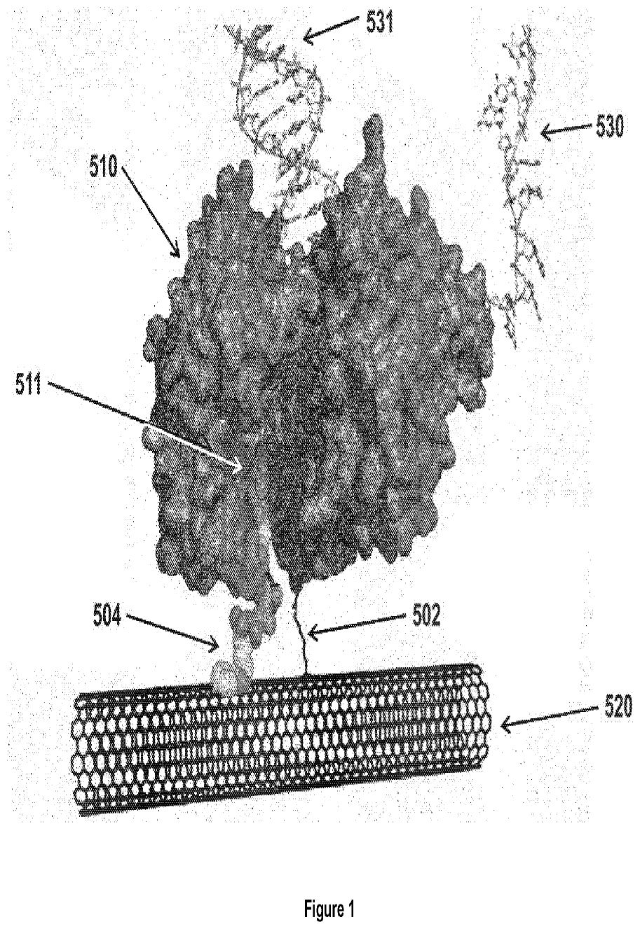

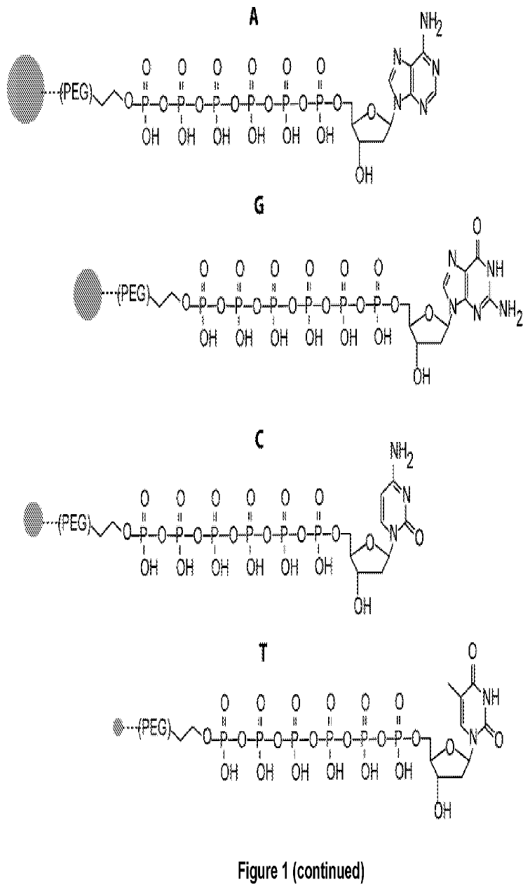

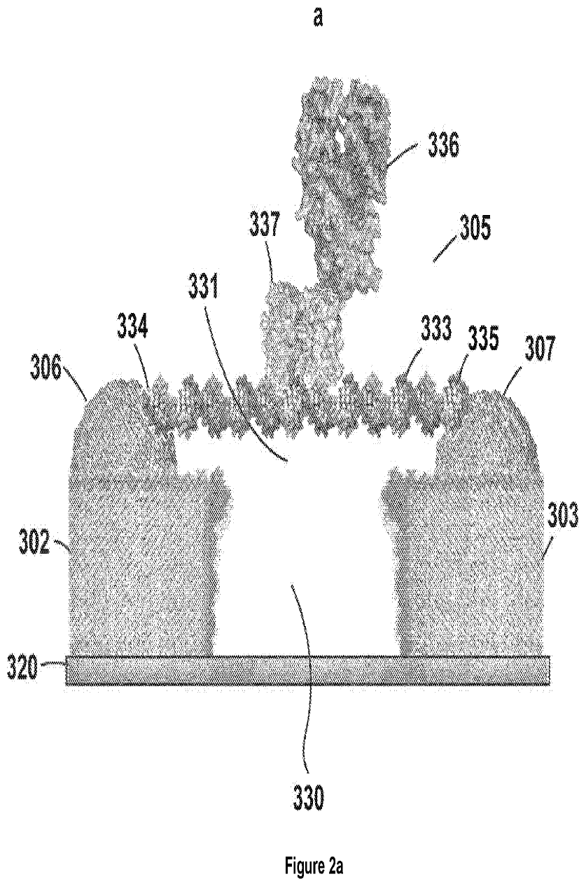

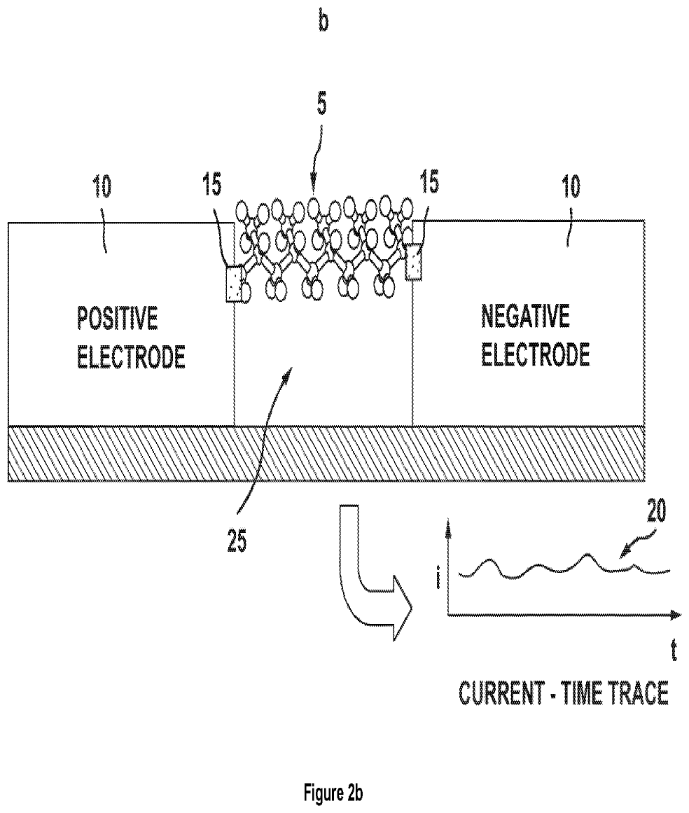

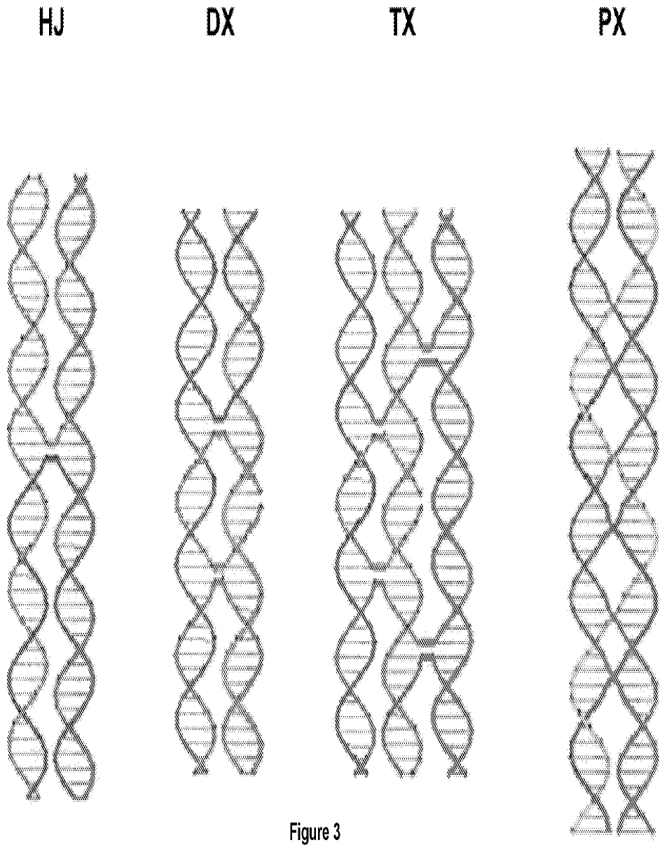

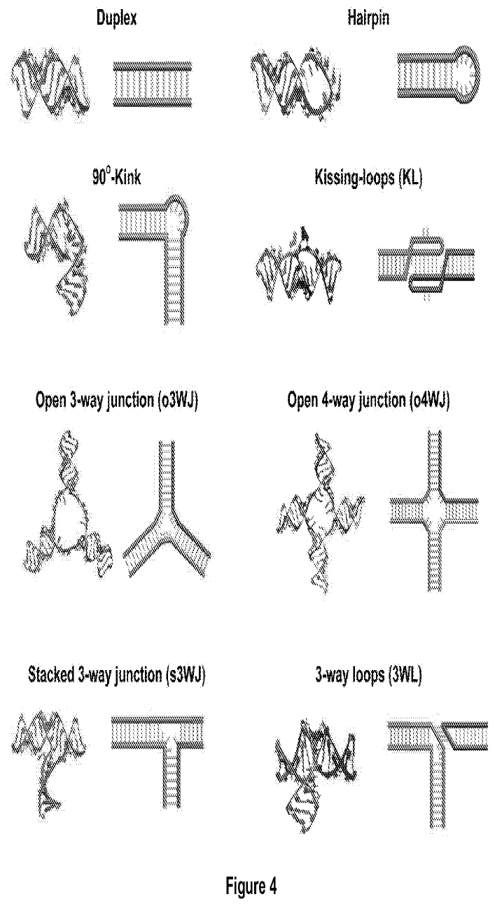

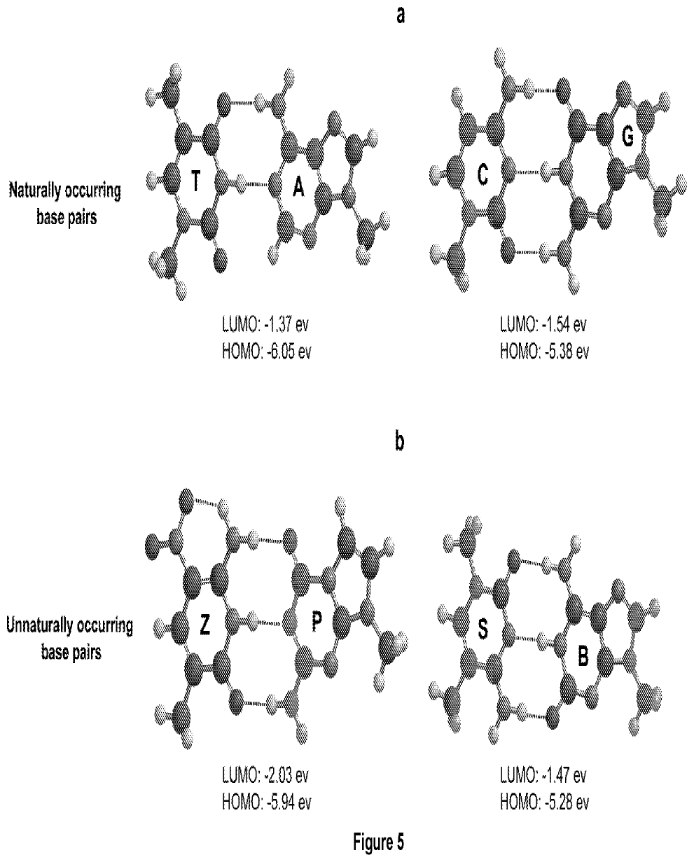

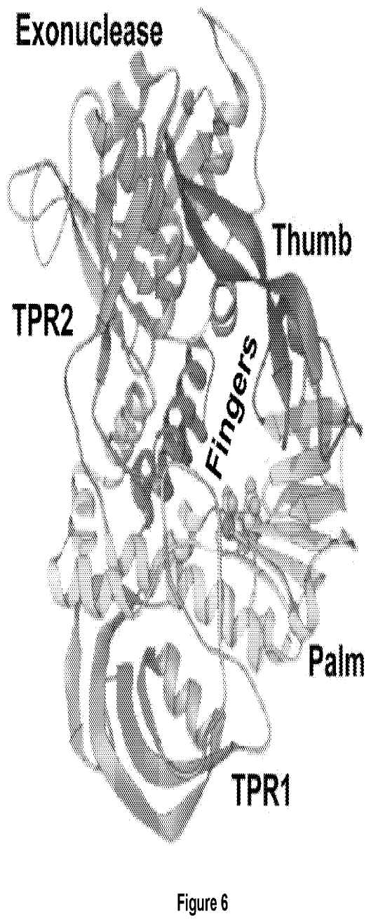

### SUMMARY OF THE INVENTION

This invention provides a nanostructure device and method for the sequencing or identification of biopolymers. This disclosure uses the sequencing of single DNA molecules to demonstrate this invention throughout the description of a variety of embodiments. This invention also provides specific technical details of a variety of representative devices, apparatus and methods in different embodiments, which are just for illustrative purpose, in no means restrict the physical dimensions and arrangement, chemical compositions and structures, processing procedures and parameters, or any other applicable conditions, and in no ways limit the scope of applications.

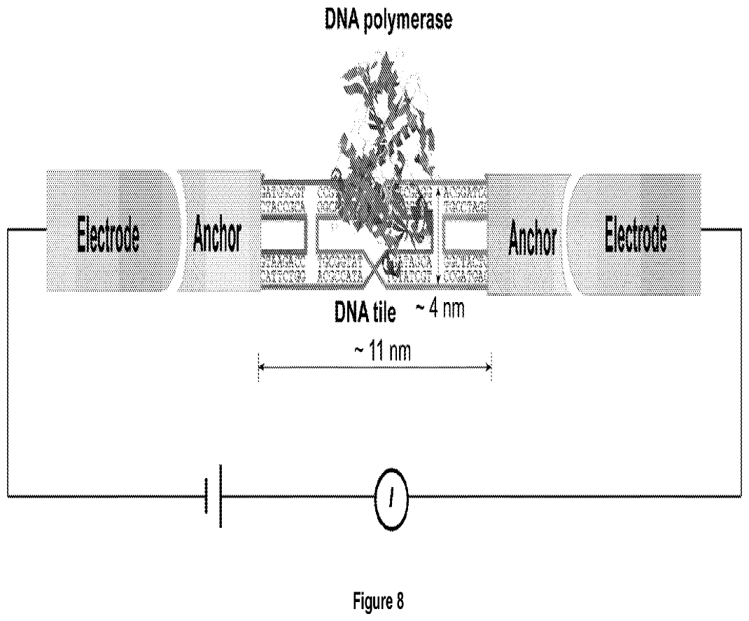

In one embodiment, as shown in FIG. 8, a 10-20 nm nanogap is fabricated by semiconductor nanofabrication technology between two electrodes with surroundings passivated with inert chemicals for the prevention of non-specific adsorption and the inner area of the nanogap exposed for the chemical reactions. A DNA tile structure is anchored to the electrodes to bridge the nanogap, on which a DNA polymerase, e.g., ϕ29 DNA polymerase, is immobilized. For sequencing, a target DNA (template) is replicated in the device. During the replicating process, nucleotides are incorporated into an elongating DNA strand by the DNA polymerase. Mechanistically, the nucleotide incorporation is accompanied by conformation changes of the polymerase (FIG. 9).⁴¹ Since the polymerase is directly attached to the DNA tile, the conformation change would disturb the tile's structure, resulting in fluctuation of electrical currents that are used as a signature to identify the incorporation of different nucleotides.

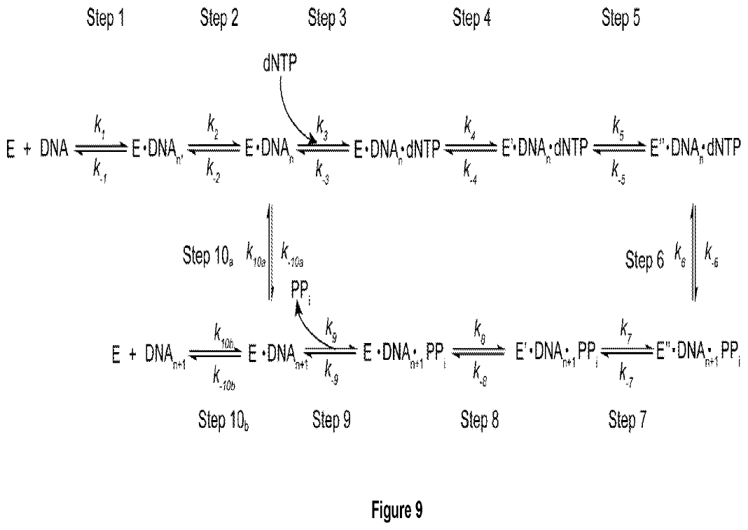

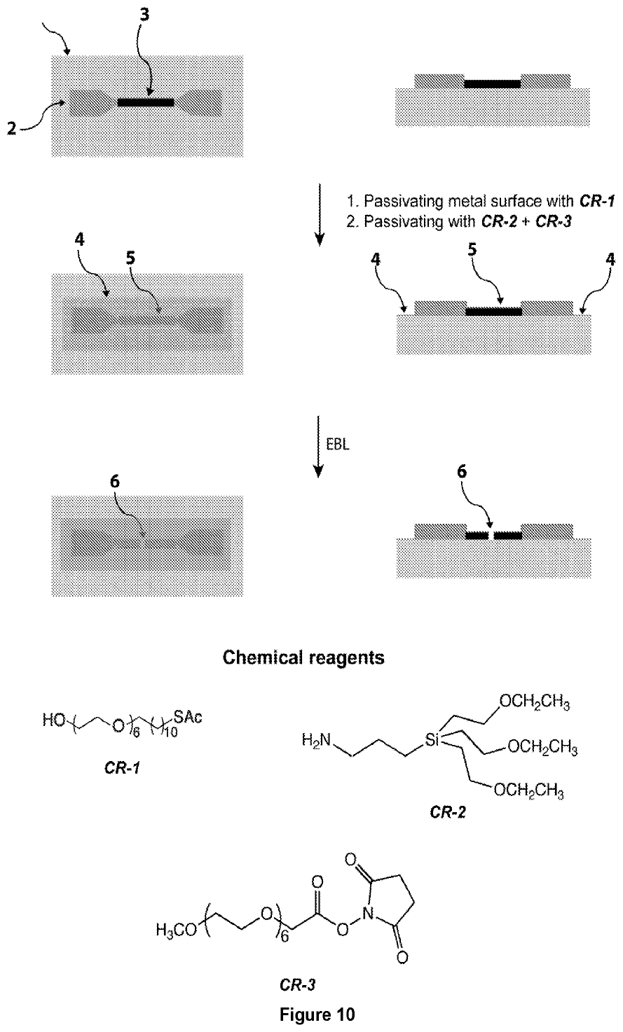

In some embodiments, the invention provides a method to fabricate a nanogap between two electrodes with a size ranging from 3 nm to 1000 nm, preferably from 5 nm to 100 nm, and more preferably from 10 nm to 30 nm. First, the invention uses electron-beam lithography (EBL) to generate metal nanowires, such as Au (gold), Pd (palladium), and Pt (platinum) nanowires over a nonconductive substrate. For example, as shown in FIG. 10, a gold nanowire (3) with a dimension of 1000×10×10 nm (Length x Width x Height) is fabricated by EBL on a silicon oxide (SiO₂) substrate (1) or a silicon substrate coated with a layer of silicon nitride (Si₃N₄), and connected to the large metal contact pads (2) by standard photolithography techniques. The length of the nanowire is between 100 nm to 100 μm, preferably 1 μm to 10 μm; the width is between 5 nm to 100 nm, preferably 10 nm to 30 nm; and the height (thickness) is between 3 nm to 100 nm, preferably 5 nm to 20 nm. An array of nanowires can also be fabricated by nanoimprinting⁴² or other nanofabrication techniques. Subsequently, the metal surface is passivated by reacting with 11-mercaptoundecyl-hexaethylene glycol (CR-1)⁴³ to form a monolayer, and the silicon oxide surface is treated first with aminopropyltriethoxysaline (CR-2), followed by reacting with N-hydroxysuccinimidyl 2-(ω-O-methoxy-hexaethylene glycol)acetate (CR-3). At last, the passivated nanowire is cut by EBL or by helium focused ion beam milling (He-FIB)⁴⁴ to generate a 10-20 nm nanogap and expose the silicon oxide and the side walls of the electrodes in the cut area. Alternatively, the nanowire or nanowires can be covered by a thin insulation layer instead of passivation or a thin insulation layer, then passivation.

In some of the embodiments, DNA nanostructures are used to bridge the nanogap. As shown in FIG. 8, a 10 nm nanogap is bridged by a four-strand DNA tile.⁴⁵ There are many methods to form DNA nanostructures with different shapes and sizes in solution through self-assembling.⁴⁶⁻⁴⁸

In one embodiment, the unnatural DNA bases (PBSZ) are used to construct the nanostructure that bridges the nanogap. It is well known that the double-helical DNA with the G/C bases is a better conductor than the one containing only A and T nucleotides. Easy oxidation of the guanine base makes it possible to generate the charge carriers (holes). The charge transport through DNA is believed to be dominated by hole transport via the base highest occupied molecular orbitals (HOMOs) because these orbitals are closer to the electrode Fermi level than the base lowest unoccupied molecular orbitals (LUMOs).⁵⁷ As shown in FIG. 5, the unnatural base pair Z:P has a HOMO with its energy higher than the one of the A:T base pair, and the base pair S:B has a HOMO with its energy higher than the one of the G:C base pair. Thus, a DNA molecule composed of these unnatural base pairs has higher conductivity than those that are composed of natural base pairs.

In one embodiment, the unnatural DNA bases (PBSZ) is used to construct conductive linear molecular wire that bridges the nanogap. The linear molecular wire is made of simple helical DNA duplex (double-strand DNA). The linear molecular wire may contain modified nucleotide(s) for the attachment or connection of polymerase or other enzymes. One benefit of using unnatural DNA bases for the construction of molecular wire is its potentially higher conductivity.

In one embodiment, the unnatural DNA bases (PBSZ) are used in the construction of more complicated conductive molecular nanostructures of either two dimensional or three dimensional, either inseparable single structure or separable multiple structure complex.

In another embodiment, the unnatural DNA bases (PBSZ) is mixed with natural bases (ACGT) to construct either simple linear conductive molecular wire or more complicated conductive molecular nanostructures that bridge the nanogap, either two dimensional or three dimensional, either inseparable single structure or separable multiple structure complex. For example, one can use natural C:G pairs plus unnatural S:B pairs to construct a DNA nanostructure to take the advantage of their high HOMO energy feature; Furthermore, one can include natural A:T pairs to form six nucleotide DNA nanostructure to employ their relatively weaker baseparing energy status to induce favorable structure changes in the DNA nanostructure. Another example is to form eight nucleotide DNA nanostructure, which is more complicated, meaning more tunable or higher probability to achieve high accuracy sequencing.

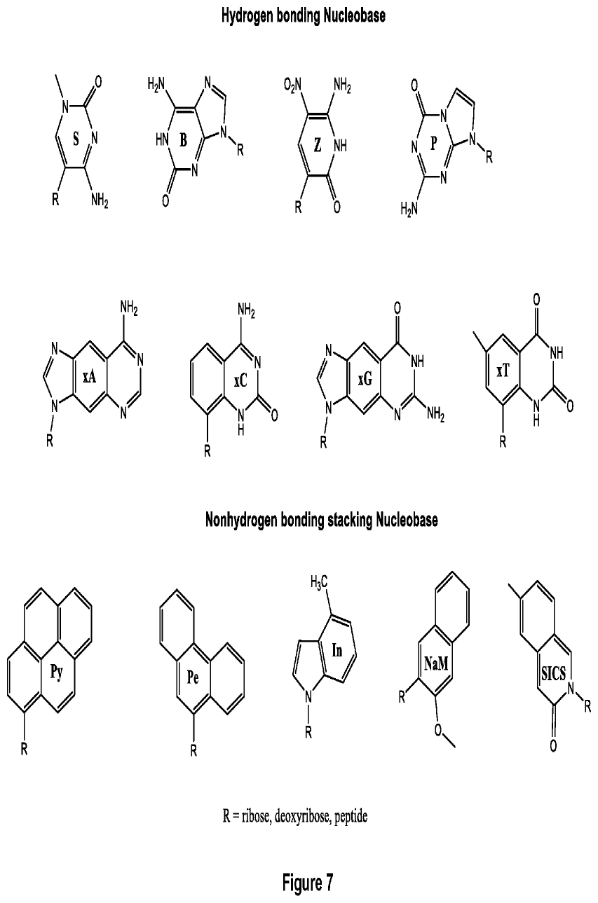

In some embodiments, this invention provides unnatural size expanded nucleic bases⁵⁸ (FIG. 7) for the formation of nucleic acid-based molecular wires (not necessarily in a helical form). Compared to the naturally occurring nucleobases, these size expanded bases possess larger π conjugation, providing better nucleobase stacking resulting in more efficient charge transport.

In some embodiments, this invention provides non-hydrogen bonding nucleobases as a part of nucleic acid-based molecular wires (FIG. 7). These nucleobases are more sensitive to changes in their surroundings, which makes the molecular wire more sensitive for bio- and chemo-sensing.

In one embodiment, this invention employs pyrene as a universal base (Py, FIG. 7), which can base pair with any of those nucleobases indiscriminately. Due to its large π conjugation, it can be inserted into the molecular wire to replace the hydrogen bonding nucleobases for increasing conductivity.

In one embodiment, unpaired or un-pairing nucleic acid base(s) can be inserted to a DNA nanostructure to purposely cause structure discontinuity in order to achieve favorable structure changes for the sequencing or identification of biopolymers.

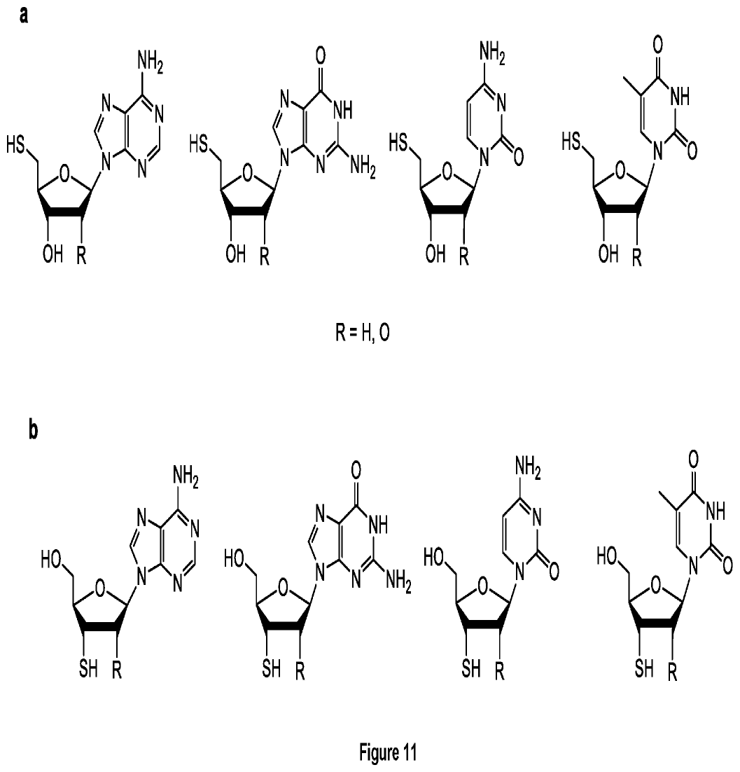

This invention also provides methods to attach the said DNA nanostructure to electrodes. In one embodiment, DNA nanostructures bear 5′-mercaptonucleosides at their 5′ ends and 3′-mercaptonucleosides at their 3′ ends, as shown in FIG. 11. The nucleosides are deoxyribonucleosides (R═H) and ribonucleosides (R═OH). Furthermore, the sulfur atom can be replaced by selenium that may be a better anchor for the electron transport.⁴⁹

In another embodiment, the invention provides methods to functionalize the DNA nanostructures at their ends with RXH and RXXR, where R is an aliphatic or aromatic group; X is chalcogens preferring to S and Se.

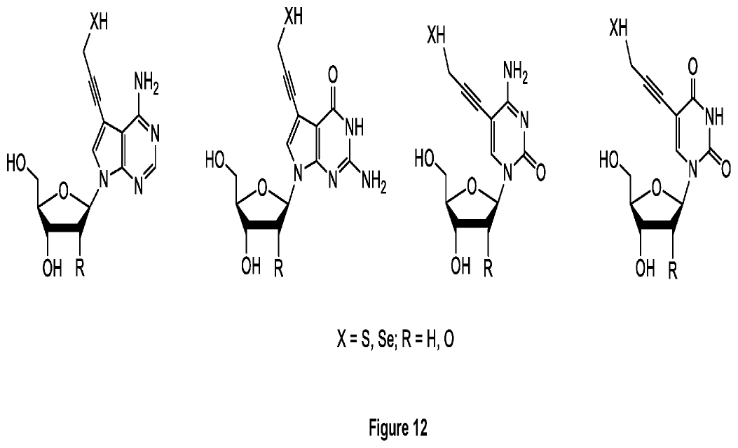

In some embodiments, the invention provides base chalcogenated nucleosides that can be incorporated into DNA nanostructures for the attachment to electrodes (FIG. 12). It has been demonstrated that connecting the electrodes DNA to electrodes via a nucleobase provides more efficient electrical contact than via the sugar moiety.⁵⁰

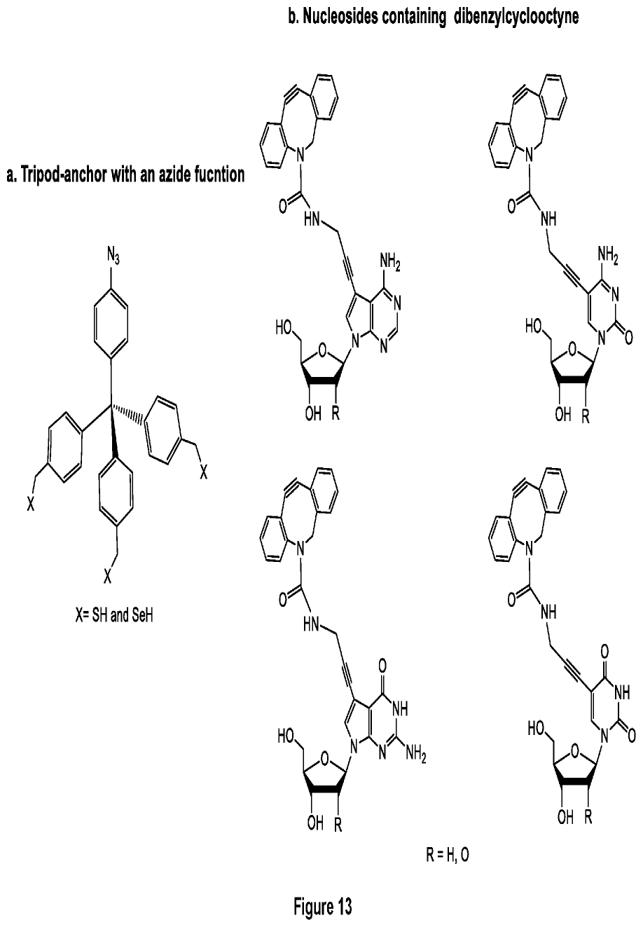

In one embodiment, the invention provides a tripod anchor comprising a tetraphenylmethane with either sulfur (S) or selenium (Se) as an anchoring atom for the attachment to metal electrodes and the carboxyl group for the attachment of a DNA nanostructure (FIG. 13, a). Meanwhile, the DNA nanostructure is modified at their ends with amino-functionalized nucleosides (FIG. 13, b) for attachment to the tripod.

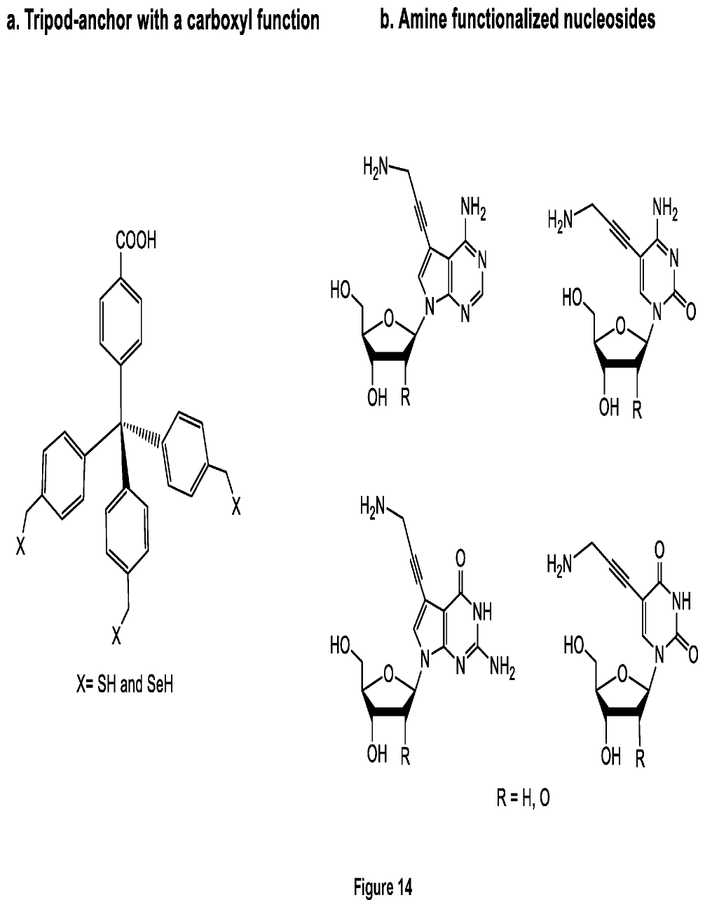

The invention also provides another tripod functionalized with azide (FIG. 14, a), which allows attaching DNA nanostructures to metal electrodes through the azide-alkyne click reactions. Therefore, the invention provides nucleosides functionalized with cyclooctyne (FIG. 14, b) for the modification of DNA nanostructures at their ends.

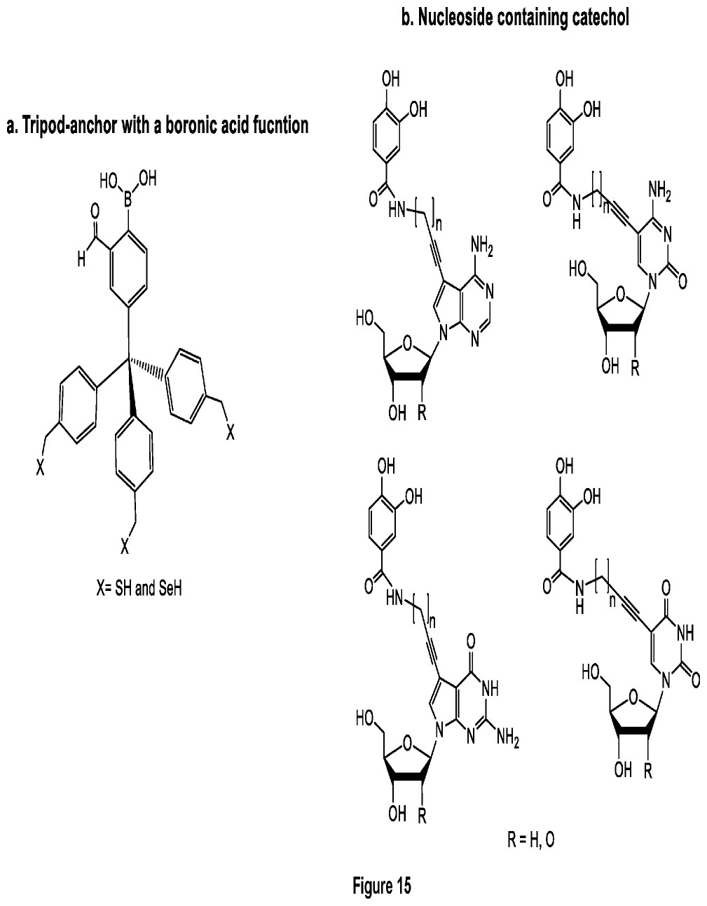

The invention also provides a tripod functionalized with boronic acid (FIG. 15, a) and nucleosides functionalized with diols (FIG. 15, b) for the modification of DNA nanostructures at their ends. Thus, a DNA nanostructure is attached to metal electrodes through the reaction of boronic acid with a diol as disclosed in the previous disclosure (Provisional patent U.S. 62/772,837).

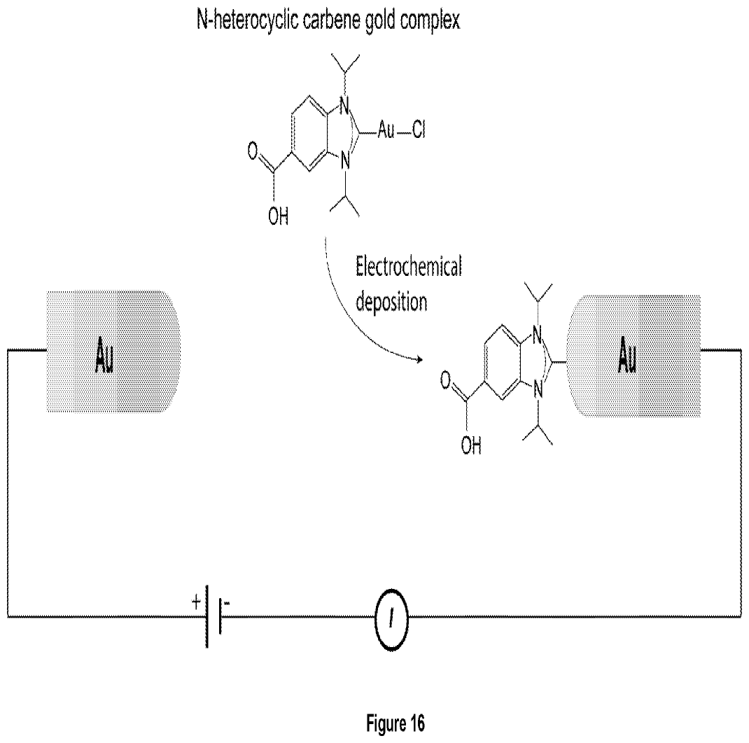

In one embodiment, the invention provides a method to selectively functionalize one of two electrodes with N-heterocyclic carbene (NHC) in a nanogap. As shown in FIG. 16, 5-carboxy-1,3-diisopropyl-1H-benzo[d]imidazol-2-carbene is deposited to a gold electrode by electrochemical reduction of its gold complex in solution.⁵¹ The carboxyl group of the NHC is used as an anchor point by converting it to an activated ester. Thus, a DNA nanostructure, with its ends functionalized respectively with amine and thiol, bridges a nanogap by its amine-functionalized end to react with the NHC electrode, and its thiol functionalized end to react with the bare gold electrode directly.

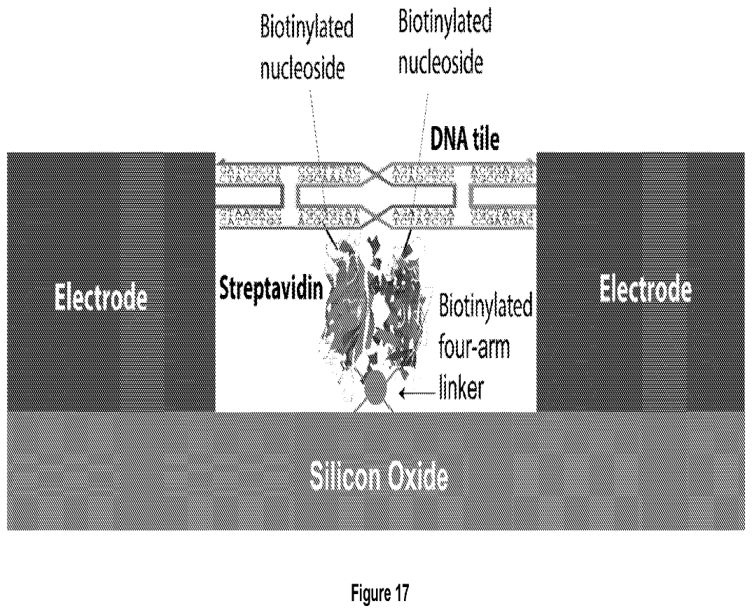

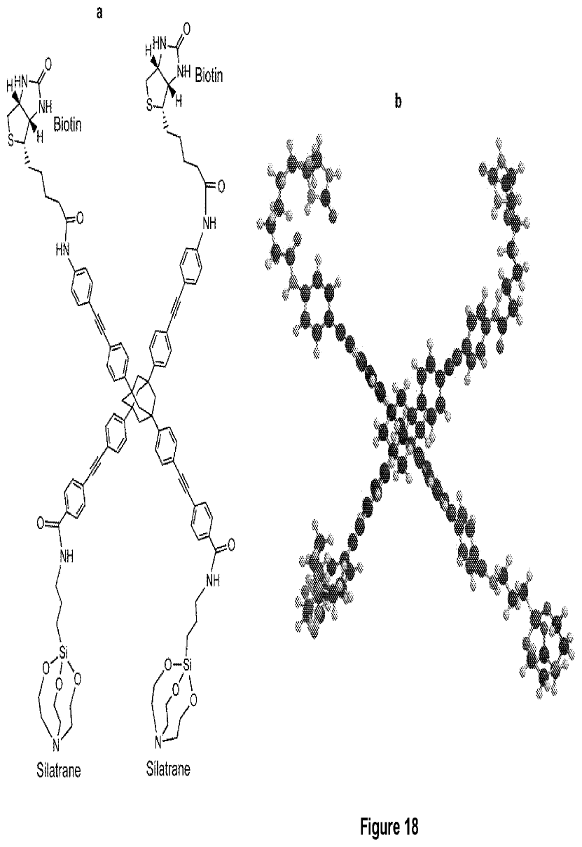

The invention provides a method to prevent a nanostructure from contacting the bottom of the nanogap. As illustrated in FIG. 17, a single streptavidin molecule is immobilized in the nanogap through a biotinylated four-arm linker so that a biotinylated DNA tile can be connected to the streptavidin, and then attached to the electrodes by one of the methods described above. The invention also provides a four-arm linker, two arms of which are functionalized with biotins and the other two with silatranes (FIG. 18, a), for the streptavidin immobilization. The four-arm linker appears to be a tetrahedron geometry by molecular mechanics' calculation (FIG. 18, b). The two biotin moieties interact with streptavidin to form a bivalent complex. For the streptavidin immobilization, the silatrane moieties first react with silicon oxide, allowing the four-arm linker to be fixed on the surface, followed by the addition of streptavidin to the surface.

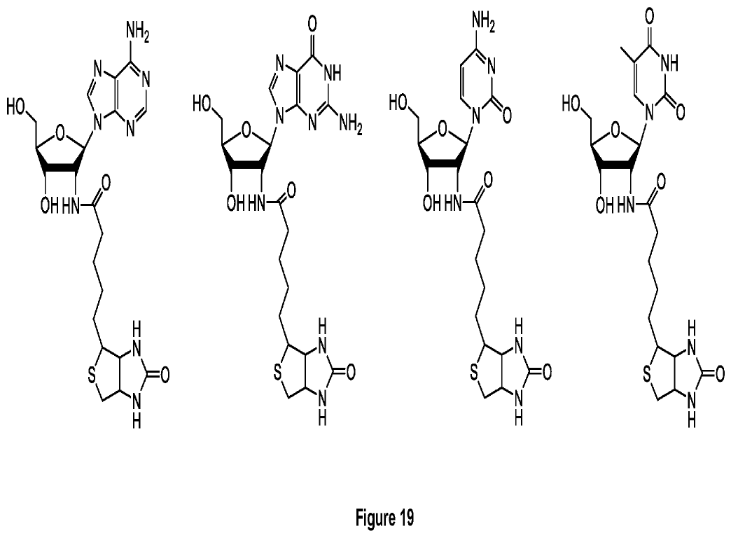

In another embodiment, the invention provides biotinylated nucleosides that can be incorporated into DNA through the phosphoramidite chemistry for the construction of DNA nanostructures (FIG. 19).

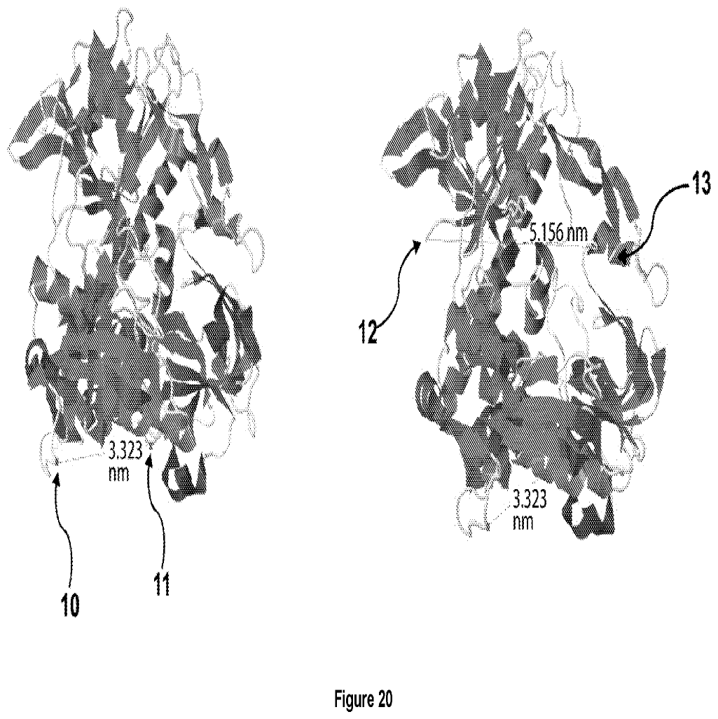

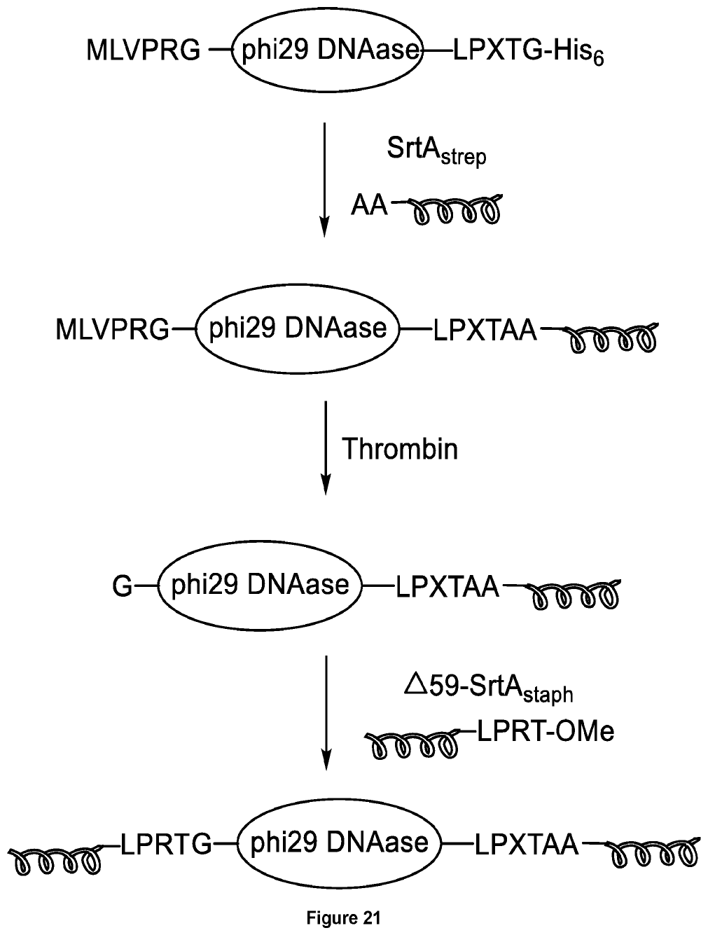

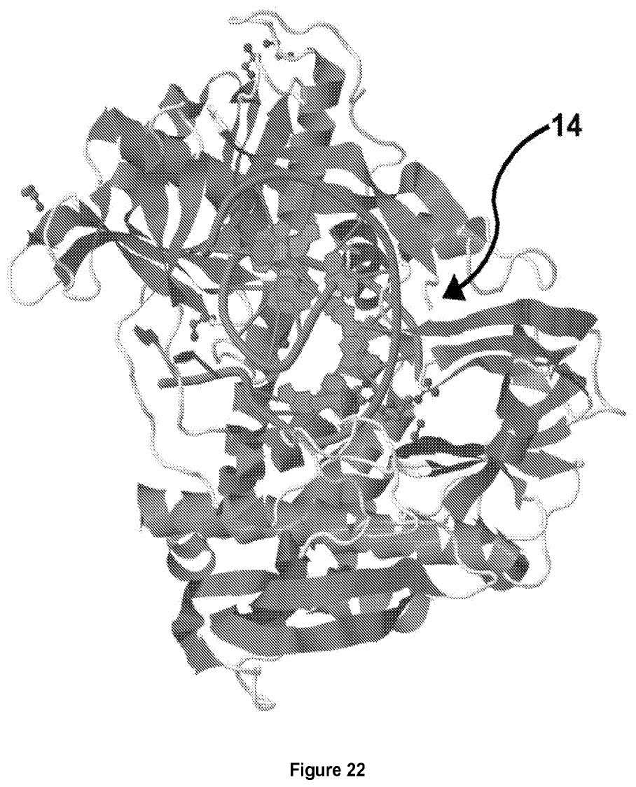

In some embodiments, the invention provides methods to attach a DNA polymerase to the DNA nanostructure. The invention employs both multi-site-directed mutagenesis method⁵² and the genetic code expansion technique⁵³ to substitute unnatural amino acids (UAAs) for canonical amino acids of the DNA polymerase at multiple specific sites. As shown in FIG. 20, a ϕ29 DNA polymerase mutant is expressed with p-azidophenylalanine substituting for W277 (10) and K479 (11). The UAA p-azidophenylalanine is used as an anchoring site for the polymerase immobilization by the click reaction, and an aaRS has already been evolved to facilitate its incorporation.^{53,54} The ϕ29 DNA polymerase mutants are further expressed to have a peptide sequence of MLVPRG at the N terminus (12) and LPXTG-His₆ at the C-terminus (13). In this way, an enzyme can be modified with peptides at its two termini. FIG. 21 shows a process of attaching peptides to the enzyme using Sortases A.⁵⁵ By viewing the structure of ϕ29 DNA polymerase complexed with primer-template DNA and incoming nucleotide substrates, one can see the C-terminus (14) of the protein is very close to the DNA (FIG. 22), suggesting that any movement of DNA in the protein could cause a domino effect on the DNA nanostructure, resulting in the fluctuations of electrical currents, which can be used as signatures of the DNA nucleotides incorporating events. Thus, fine-tuning the DNA nanostructure can achieve single-base resolution.

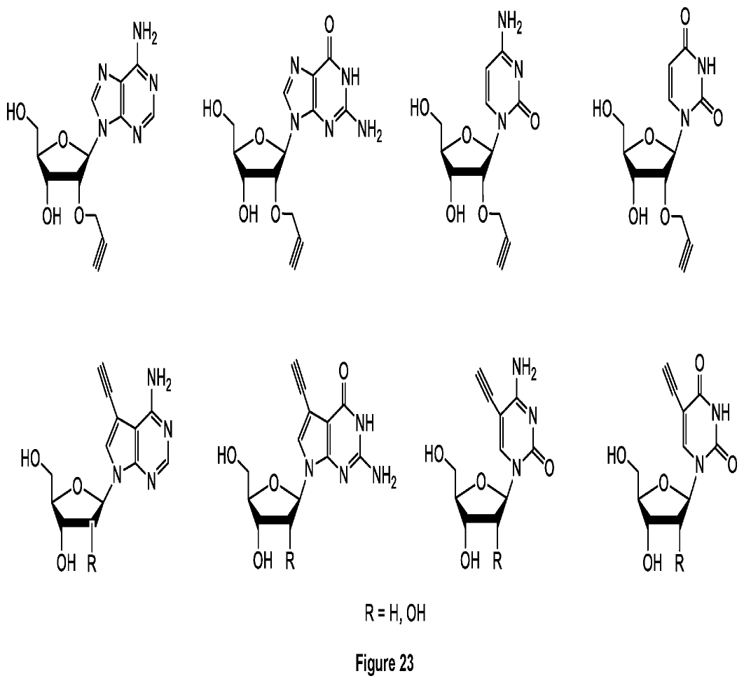

In one embodiment, the invention provides nucleosides containing acetylene that can be incorporated into DNA for the construction of DNA nanostructures for attaching the DNA polymerase through the click reaction in the presence of a copper catalyst (FIG. 23).

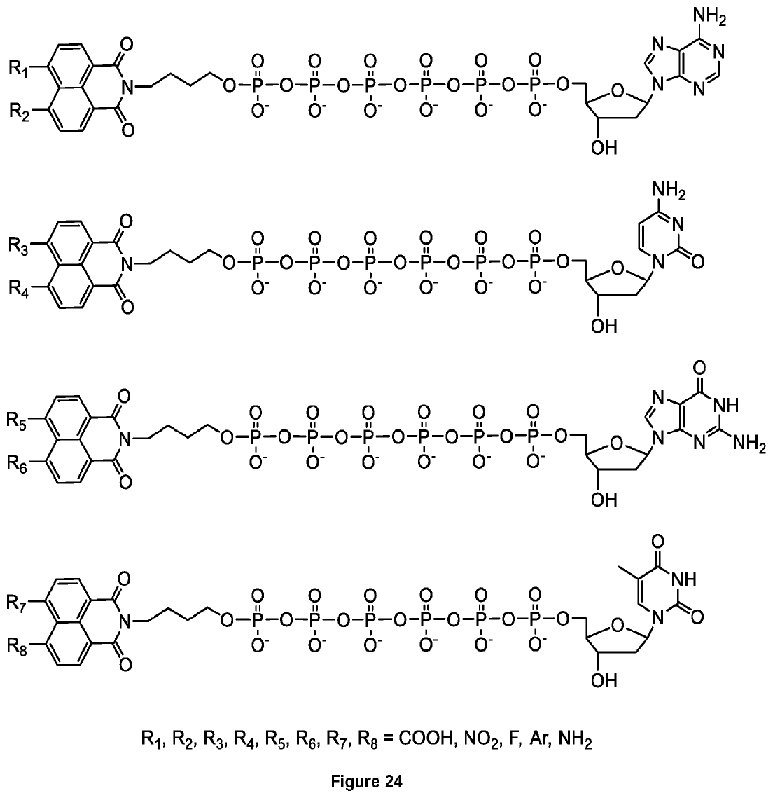

In one embodiment, the invention provides modified nucleotides (dN6P) tagged with different DNA intercalators that interact with DNA nanostructures (FIG. 24). These modified nucleotides are used as substrates for a DNA polymerase to incorporate DNA nucleotides into DNA. First, the DNA polymerase forms a complex with a target DNA template and a nucleoside polyphosphate, which also stabilizes the interaction of the intercalator tag with the DNA nanostructure. When the nucleotide is incorporated into target DNA, it releases a pentaphosphate tagged with an intercalator. Because the electrostatic repulsion destabilizes the interaction of intercalator with DNA, it results in the release of the tagged pentaphosphate into solution. Such a process would change the conductance of the DNA nanostructure. Since each dN6P carries a different intercalator, the incorporation of a different nucleotide would cause different current fluctuations, which can be used to identify the nucleotide incorporated into DNA.

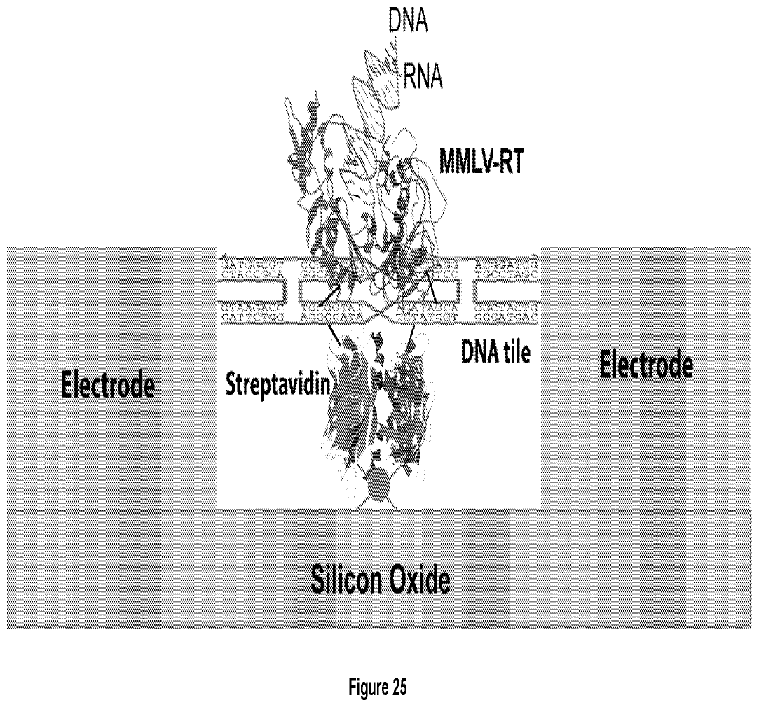

Most of the methods in the above-disclosed embodiments apply to RNA sequencing. In one embodiment, a re-engineered Moloney Murine Leukemia Virus Reverse Transcriptase (M-MLV RT) is immobilized on the DNA tile for the RNA reverse transcription, as shown in FIG. 25. When an RNA target primed with poly(dT) is introduced into the device, DNA nucleotides are incorporated into the poly(dT) primer. In this process, each incorporation causes changes of the poiymerase's conformation, resulting in fluctuations of electrical currents. With the incorporation continuation, a train of electric signals is recorded, from which the RNA sequence is deduced with an analytical program.

### GENERAL REMARKS

All publications, patents and other documents mentioned herein are incorporated by reference in their entirety.

Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will be readily appear to those skilled in the art. The invention in its broader aspects is therefore not limited to the specific details, representative device, apparatus and method, and illustrative example shown and described. Accordingly, departures may be made from such details without departing from the spirit of applicant's general inventive concept.

### REFERENCES

1. Sanger, F.; Coulson, A. R., A rapid method for determining sequences in DNA by primed synthesis with DNA polymerase. J. Mol. Biol. 1975, 95, 441-448.

2. Sanger, F.; Nicklen, S.; Coulson, A. R., DNA sequencing with chain-terminating inhibitors. Proc. Natl. Acad. Sci. USA 1977, 74, 5463-5467.

3. Chen, C. Y., DNA polymerases drive DNA sequencing-by-synthesis technologies: both past and present. Front. Microbiol. 2014, 5, 305.

4. Mardis, E. R., DNA sequencing technologies: 2006-2016. Nat. Protoc. 2017, 12, 213-218.

5. Pourmand, N.; Karhanek, M.; Persson, H. H. J.; Webb, C. D.; Lee, T. H.; Zahradni‘kova’, A.; Davis, R. W., Direct electrical detection of DNA synthesis. Proc. Nati. Acad. Sci. USA 2006, 103, 6466-6470.

6. Rothberg, J. M.; Hinz, W.; Rearick, T. M.; Schultz, J.; Mileski, W.; Davey, M.; Leamon, J. H.; Johnson, K.; Milgrew, M. J.; Edwards, M.; Hoon, J.; Simons, J. F.; Marran, D.; Myers, J. W.; Davidson, J. F.; Branting, A.; Nobile, J. R.; Puc, B. P.; Light, D.; Clark, T. A.; Huber, M.; Branciforte, J. T.; Stoner, I. B.; Cawley, S. E.; Lyons, M.; Fu, Y.; Homer, N.; Sedova, M.; Miao, X.; Reed, B.; Sabina, J.; Feierstein, E.; Schorn, M.; Alanjary, M.; Dimalanta, E.; Dressman, D.; Kasinskas, R.; Sokolsky, T.; Fidanza, J. A.; Namsaraev, E.; McKernan, K. J.; Williams, A.; Roth, G. T.; Bustillo, J., An integrated semiconductor device enabling non-optical genome sequencing. Nature 2011, 475 (7356), 348-52.

7. Cherf, G. M.; Lieberman, K. R.; Rashid, H.; Lam, C. E.; Karplus, K.; Akeson, M., Automated forward and reverse ratcheting of DNA in a nanopore at 5-A precision. Nat. Biotechnol. 2012, 30, 344-8.

8. Jain, M.; Fiddes, I. T.; Miga, K. H.; Olsen, H. E.; Paten, B.; Akeson, M., Improved data analysis for the MinION nanopore sequencer. Nat. methods 2015, 12, 351-6.

9. Manrao, E. A.; Derrington, I. M.; Laszlo, A. H.; Langford, K. W.; Hopper, M. K.; Gillgren, N.; Pavlenok, M.; Niederweis, M.; Gundlach, J. H., Reading DNA at single-nucleotide resolution with a mutant MspA nanopore and phi29 DNA polymerase. Nat. Biotechnol. 2012, 30, 349-353.

10. Laszlo, A. H.; Derrington, I. M.; Ross, B. C.; Brinkerhoff, H.; Adey, A.; Nova, I. C.; Craig, J. M.; Langford, K. W.; Samson, J. M.; Daza, R.; Doering, K.; Shendure, J.; Gundlach, J. H., Decoding long nanopore sequencing reads of natural DNA. Nat. Biotechnol. 2014, 32, 829-833.

11. Lindsay, S., The promises and challenges of solid-state sequencing. Nat Nanotechnol 2016, 11 (2), 109-11.

12. Olsen, T. J.; Choi, Y.; Sims, P. C.; Gul, 0. T.; Corso, B. L.; Dong, C.; Brown, W. A.; Collins, P. G.; Weiss, G. A., Electronic measurements of single-molecule processing by DNA polymerase I (Klenow fragment). J. Am. Chem. Soc. 2013, 135, 7855-60.

13. Pugliese, K. M.; Gul, O. T.; Choi, Y.; Olsen, T. J.; Sims, P. C.; Collins, P. G.; Weiss, G. A., Processive Incorporation of Deoxynucleoside Triphosphate Analogs by Single-Molecule DNA Polymerase I (Klenow Fragment) Nanocircuits. J. Am. Chem. Soc. 2015, 137, 9587-94.

14. Choi, Y.; Olsen, T. J.; Sims, P. C.; Moody, I. S.; Corso, B. L.; Dang, M. N.; Weiss, G. A.; Collins, P. G., Dissecting Single-Molecule Signal Transduction in Carbon Nanotube Circuits with Protein Engineering. Nano Lett. 2013, 13, 625-631.

15. Xavier, P. L.; Chandrasekaran, A. R., DNA-based construction at the nanoscale: emerging trends and applications. Nanotechnology 2018, 29, 062001.

16. Ge, Z.; Gu, H.; Li, Q.; Fan, C., Concept and Development of Framework Nucleic Acids. J. Am. Chem. Soc. 2018, 140, 17808-17819.

17. Sha, R.; Liu, F.; Seeman, N. C., Atomic Force Microscopic Measurement of the Interdomain Angle in Symmetric Holliday Junctions. Biochemistry 2002, 41, 5950-5955.

18. Mao, C.; Sun, W.; Seeman, N. C., Designed Two-Dimensional DNA Holliday Junction Arrays Visualized by Atomic Force Microscopy. J. Am. Chem. Soc. 1999, 121, 5437-5443.

19. Wang, X.; Seeman, N. C., Assembly and Characterization of 8-Arm and 12-Arm DNA Branched Junctions. J. Am. Chem. Soc. 2007, 129, 8169-8176.

20. Fu, T.-J.; Seeman, N. C., DNA Double-Crossover Molecules. Biochemistry & Analytical Biochemistry 1993, 32, 3211-3220.

21. LaBean, T. H.; Yan, H.; Kopatsch, J.; Liu, F.; Winfree, E.; Reif, J. H.; Seeman, N. C., Construction, Analysis, Ligation, and Self-Assembly of DNA Triple Crossover Complexes. J. Am. Chem. Soc. 2000, 122, 1848-1860.

22. Wang, X.; Chandrasekaran, A. R.; Shen, Z.; Ohayon, Y. P.; Wang, T.; Kizer, M. E.; Sha, R.; Mao, C.; Yan, H.; Zhang, X.; Liao, S.; Ding, B.; Chakraborty, B.; Jonoska, N.; Niu, D.; Gu, H.; Chao, J.; Gao, X.; Li, Y.; Ciengshin, T.; Seeman, N. C., Paranemic Crossover DNA: There and Back Again. Chem. Rev. 2018, 119, 6273-6289.

23. Liu, D.; Wang, M.; Deng, Z.; Walulu, R.; Mao, C., Tensegrity: Construction of Rigid DNA Triangles with Flexible Four-Arm DNA Junctions. J. Am. Chem. Soc. 2004, 126, 2324-2325.

24. Mathieu, F.; Liao, S.; Kopatsch, J.; Wang, T.; Mao, C.; Seeman, N. C., Six-Helix Bundles Designed from DNA. Nano Lett. 2005, 5, 661-665.

25. Rothemund, P. W., Folding DNA to create nanoscale shapes and patterns. Nature 2006, 440, 297-302.

26. Linko, V.; Leppiniemi, J.; Paasonen, S. T.; Hytonen, V. P.; Toppari, J. J., Defined-size DNA triple crossover construct for molecular electronics: modification, positioning and conductance properties. Nanotechnology 2011, 22, 275610.

27. Grabow, W. W.; Jaeger, L., RNA self-assembly and RNA nanotechnology. Acc. Chem. Res. 2014, 47, 1871-80.

28. Li, M.; Zheng, M.; Wu, S.; Tian, C.; Liu, D.; Weizmann, Y.; Jiang, W.; Wang, G.; Mao, C., In vivo production of RNA nanostructures via programmed folding of single-stranded RNAs. Nat. Commun. 2018, 9, 2196.

29. Maie, K.; Miyagi, K.; Takada, T.; Nakamura, M.; Yamana, K., RNA-Mediated Electron Transfer: Double Exponential Distance Dependence. J. Am. Chem. Soc.. 2009, 131, 13188-13189.

30. Gao, X.; Gethers, M.; Han, S. P.; Goddard, W. A.; Sha, R.; Cunningham, R.; Seeman, N. C., The PX-Motif of DNA Binds Specifically to E. coli DNA Polymerase I. Biochemistry 2019, 58, 575-581

31. Korlach, J.; Marks, P. J.; Cicero, R. L.; Gray, J. J.; Murphy, D. L.; Roitman, D. B.; Pham, T. T.; Otto, G. A.; Foquet, M.; Turner, S. W., Selective aluminum passivation for targeted immobilization of single DNA polymerase molecules in zero-mode waveguide nanostructures. Proc. Nati. Acad. Sci. USA 2008, 105, 1176-81.

32. Stranges, P. B.; Palla, M.; Kalachikov, S.; Nivala, J.; Dorwart, M.; Trans, A.; Kumar, S.; Porel, M.; Chien, M.; Tao, C.; Morozova, I.; Li, Z.; Shi, S.; Aberra, A.; Arnold, C.; Yang, A.; Aguirre, A.; Harada, E. T.; Korenblum, D.; Pollard, J.; Bhat, A.; Gremyachinskiy, D.; Bibillo, A.; Chen, R.; Davis, R.; Russo, J. J.; Fuller, C. W.; Roever, S.; Ju, J.; Church, G. M., Design and characterization of a nanopore-coupled polymerase for single-molecule DNA sequencing by synthesis on an electrode array. Proc. Nati. Acad. ScL USA 2016, 113, E6749-E6756.

33. Bernad, A.; Zabalios, A.; Salas, M.; Blanco, L., Structural and functional relationships between prokaryotic and eukaryotic DNA polymerases. The EMBO journal 1987, 6, 4219-4225.

34. Esteban, J. A.; Salas, M.; Blanco, L., Fidelity of phi29 DNA Polymerase. J. Bio. Chem. 1993, 268, 2713-2726.

35. Blanco, L.; Salas, M., Characterization of a 3′-5′ exonuclease activity in the phage phi29-encoded DNA polymerase. Nucleic Acids Research 1985, 13, 1239-1249.

36. Garmendia, C.; Bernad, A.; Estebane, J. A.; Blanco, L.; Salad, M., The Bacteriophage phi29 DNA Polymerase, a Proofreading Enzyme. J. Bio. Chem. 1992, 267, 2594-2599.

37. Esteban, J. A.; Soengas, M. S.; Salasn, M.; Blanco, L., 3′ to 5′ Exonuclease Active Site of 429 DNA Polymerase. J. Bio. Chem. 1994, 269, 31946-31954.

38. Kamtekar, S.; Berman, A. J.; Wang, J.; Lazaro, J. M.; de Vega, M.; Blanco, L.; Salas, M.; Steitz, T. A., Insights into strand displacement and processivity from the crystal structure of the protein-primed DNA polymerase of bacteriophage phi29. Molecular cell 2004, 16, 609-18.

39. Berman, A. J.; Kamtekar, S.; Goodman, J. L.; Lazaro, J. M.; Vega, M. d.; Blanco, L.; Salas, M.; Steitz, T. A., Structures of phi29 DNA polymerase complexed with substrate: the mechanism of translocation in B-family polymerases. The EMBO journal 2007, 26, 3494-3505 I.

40. Rodn'guez, I.; zaro, J. M. L.; Blanco, L.; Kamtekar, S.; Berman, A. J.; Wang, J.; Steitz, T. A.; Salas, M.; Vega, M. d., A specific subdomain in □29 DNA polymerase confers both processivity and strand-displacement capacity. Proc. Nati. Acad. Sci. USA 2005, 102, 6407-6412.

41. Raper, A. T.; Reed, A. J.; Suo, Z., Kinetic Mechanism of DNA Polymerases: Contributions of Conformational Dynamics and a Third Divalent Metal Ion. Chem. Rev. 2018, 118, 6000-6025.

42. Pi, S.; Lin, P.; Xia, Q., Fabrication of sub-10 nm metal nanowire arrays with sub-1 nm critical dimension control. Nanotechnology 2016, 27, 464004.

43. Senapati, S.; Biswas, S.; Manna, S.; Ros, R.; Lindsay, S.; Zhang, P., A Y-Shaped Three-Arm Structure for Probing Bivalent Interactions between Protein Receptor-Ligand Using AFM and SPR. Langmuir 2018, 34, 6930-6940.

44. Scholder, O.; Jefimovs, K.; Shorubalko, I.; Hafner, C.; Sennhauser, U.; Bona, G. L., Helium focused ion beam fabricated plasmonic antennas with sub-5 nm gaps. Nanotechnology 2013, 24, 395301.

45. Evans, C. G.; Winfree, E., Physical principles for DNA tile self-assembly. Chem. Soc. Rev. 2017, 46, 3808-3829.

46. Endo, M.; Sugiyama, H., Chemical approaches to DNA nanotechnology. Chembiochem 2009, 10, 2420-43.

47. Hong, F.; Jiang, S.; Lan, X.; Narayanan, R. P.; Sulc, P.; Zhang, F.; Liu, Y.; Yan, H., Layered-Crossover Tiles with Precisely Tunable Angles for 2D and 3D DNA Crystal Engineering. J. Am. Chem. Soc. 2018, 140, 14670-14676.

48. Wang, W.; Lin, T.; Zhang, S.; Bai, T.; Mi, Y.; Wei, B., Self-assembly of fully addressable DNA nanostructures from double crossover tiles. Nucleic Acids Res 2016, 44, 7989-96.

49. Kaull, R. P.; Sawhne, R. S.; Kaul, R.; Engles#, D., Single-Molecule Junctions Based on Selenol Terminated Anchor-A Promising Candidate for Electron Transport. proceedings of the International Conference on Advanced Nanomaterials & Emerging Engineering Technologies 2013, ICANMEET-20J3, 431-435.

50. Xiang, L.; Palma, J. L.; Bruot, C.; Mujica, V.; Ratner, M. A.; Tao, N., Intermediate tunnelling-hopping regime in DNA charge transport. Nat. Chem. 2015, 7, 221-6.

51. Doud, E. A.; lnkpen, M. S.; Lovat, G.; Montes, E.; Paley, D. W.; Steigerwald, M. L.; Vazquez, H.; Venkataraman, L.; Roy, X., In Situ Formation of N-Heterocyclic Carbene-Bound Single-Molecule Junctions. J. Am. Chem. Soc. 2018, 140, 8944-8949.

52. Liang, X.; Peng, L.; Li, K.; Peterson, T.; Katzen, F., A method for multi-site-directed mutagenesis based on homologous recombination. Anal. Biochem. 2012, 427, 99-101.

53. Chatterjee, A.; Sun, S. B.; Furman, J. L.; Xiao, H.; Schultz, P. G., A versatile platform for single- and multiple-unnatural amino acid mutagenesis in Escherichia coli. Biochemistry 2013, 52, 1828-37.

54. Chin, J. W.; Santoro, S. W.; Martin, A. B.; King, D. S.; Wang, L.; Schultz, P. G., Addition of p-Azido-L-phenylalanine to the Genetic Code of Escherichia coli. J. Am. Chem. Soc. 2002, 124, 9026-9027.

55. Antos, J. M.; Chew, G.-L.; Guimaraes, C. P.; Yoder, N. C.; Grotenbreg, G. M.; Popp, M. W.-L.; Ploegh, H. L., Site-Specific N- and C-Terminal Labeling of a Single Polypeptide Using Sortases of Different Specificity. J. Am. Chem. Soc. 2009, 131, 10800-10801.

55. Ishiguro, T., Yamaji, K. and Saito, G., Organic Superconductors, Springer Series in Solid-State Sciences, 2nd Edition, ISSN 0171-1873, 1998.

56. Hoshika, S.; Leal, N. A.; Kim, M.-J.; Kim, M.-S.; Karalkar, N. B.; Kim, H.-J.; Bates, A. M.; Jr., N. E. W.; SantaLucia, H. A.; Meyer, A. J.; DasGupta, S.; Piccirilli, J. A.; Ellington, A. D.; Jr., J. S.; Georgiadis, M. M.; Benner, S. A., Hachimoji DNA and RNA: A genetic system with eight building blocks. Science 2019, 363, 884-887

57. Xiang, L.; Palma, J. L.; Bruot, C.; Mujica, V.; Ratner, M. A.; Tao, N., Intermediate tunnelling-hopping regime in DNA charge transport. Nat Chem 2015, 7, 221-6.

58. Winnacker, M.; Kool, E. T., Artificial genetic sets composed of size-expanded base pairs. Angew Chem Int Ed Engl 2013, 52, 12498-508.

What is claimed is:

1. A system for identification, characterization, or sequencing of a biopolymer comprising,
(a) a non-conductive substrate;
(b) a nanogap formed by a first electrode and a second electrode placed next to each other on the non-conductive substrate;
(c) a nanostructure that bridges the nanogap by attaching one end to the first electrode and another end to the second electrode through a chemical bond, wherein the nanostructure comprises a nucleic acid, either deoxyribonucleic acid (DNA nanostructure) or ribonucleic acid (RNA nanostructure) or a combination thereof, wherein a base of the nucleic acid are either an unnatural nucleic acid base or the nucleic acid comprises a mixture of unnatural and natural nucleic acid bases;
(d) an enzyme attached to the nanostructure that performs a biochemical reaction;
(e) a bias voltage that is applied between the first electrode and the second electrode;
(f) a device that records a current fluctuation through the nanostructure resulting from a distortion within the nanostructure caused by a conformation change initiated by the enzyme attached to the nanostructure; and
(g) a software for data analysis that identifies or characterizes the biopolymer or a subunit of the biopolymer.

2. The system of claim 1, wherein the non-conductive substrate is selected from the group consisting of a silicon, a silicon oxide, a silicon a nitride, a glass, a hafnium dioxide, an other metal oxides, any non-conductive polymer film, a silicon with silicon oxide or silicon nitride or other non-conductive coating, a glass with silicon nitride coating, an other non-conductive organic material, any non-conductive inorganic materials, and a combination thereof.

3. The system of claim 1, wherein the biopolymer is selected from the group consisting of a DNA, a RNA, an oligonucleotide, a protein, a peptide, a polysaccharide, either natural, modified or synthesized of any of the aforementioned biopolymers, and a combination thereof.

4. The system of claim 1, wherein the enzyme is selected from the group consisting of a DNA polymerase, a RNA polymerase, a DNA helicase, a DNA ligase, a DNA exonuclease, a reverse transcriptase, a RNA primase, a ribosome, a sucrase, a lactase, either native, mutated, expressed, or synthesized of any of the aforementioned enzymes, and a combination thereof.

5. The system of claim 4, wherein the enzyme is selected from the group consisting of T7 DNA polymerase, Taq polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol I (iota), Pol κ (kappa), pol η (eta), terminal deoxynucleotidyl transferase, telomerase, either native, mutated, expressed, or synthesized of any of the aforementioned enzymes, and a combination thereof .

6. The system of claim 4, wherein the DNA polymerase is ϕ29 DNA polymerase, either native, mutated, expressed, or synthesized.

7. The system of claim 1,
wherein the two electrodes forming the nanogap are separated by a distance of about 3 nm to about 1000 nm; and
wherein the ends of the electrodes have a substantially rectangular face with a width of about 3 nm to about 1000 nm, and a depth of about 2 nm to about 1000 nm.

8. The system of claim 1,
wherein the two electrodes forming the nanogap are separated by a distance of about 5 nm to about 50 nm; and
wherein the ends of the electrodes have a substantial rectangular face with a width of about 10 nm to about 30 nm, and a depth of about 5 nm to about 20 nm.

9. The system of claim 1, wherein the electrode comprises:
a) a metal electrode that can react with a thiol, a amine, a selenol, and another organic functional group;
b) a metal electrode that can be functionalized on the surface by a self-assembling monolayer that can react with an anchoring molecule to form a covalent bond;
c) a metal oxide electrode that can be functionalized with a silane that can react with the anchoring molecule to form a covalent bond; or
d) a carbon electrode that can be functionalized with an organic reagentthat can react with the anchoring molecule to form a covalent bond.

10. The system of claim 1, wherein the electrode and the substrate are passivated with an insulating layer except for the end surface that faces the nanogap.

11. The system of claim 10, wherein the insulation layer either comprises a monolayer or multi-layers of an inert chemicals or is passivated by a monolayer or multi-layers of an inert chemical.

12. The system of claim 11, wherein the inert chemical comprises an 11-mercaptoundecyl-hexaethylene glycol (CR-1) fora metal surface passivation, and an aminopropyltriethoxysaline (CR-2) & N-hydroxysuccinimidyl 2-(ω-O-methoxy-hexaethylene glycol)acetate (CR-3) for the substrate surface passivation.

13. The system of claim 1, wherein the unnatural nucleic acid base is selected from the group consisting of
(a) a Hachimoji nucleic acid base;
(b) a size expanded nucleobase;
(c) a non-hydrogen bonding nucleobase;
(d) an universal base; and
(e) a combination thereof.

14. The system of claim 1, wherein the nucleic acid nanostructure comprises one of the following or a combination thereof:
(a) an unpaired or unpairing nucleic acid base; or
(b) an organic superconductor.

15. The system of claim 1, wherein
the nucleic acid nanostructure is self-assembled from either linear or circular DNAs (DNA nanostructure), or linear or circular RNAs (RNA nanostructure), or a combination thereof.

16. The system of claim 1, wherein
the nucleic acid nanostructure has one of the following configurations or a combination thereof:
(a) a substantially one-dimensional geometry, including but not limited to, a linear DNA duplex structure, a linear RNA duplex structure, a linear nucleic acid molecular wire either in helical form or non-helical form, or a combination thereof;
(b) a substantially two-dimensional geometry, including but not limited to, a substantially rectangular structure, a substantially square structure, a substantially triangular structure, a substantially circular structure, or a combination thereof; and
(c) a substantially three-dimensional geometry, including but not limited to, a substantially cylindrical structure, a substantially hollow tube structure, a substantially column-like structure, a geometry comprising a substantially bundle-of-columns structure, a geometry comprising a substantially stacked two-dimensional structure, a geometry comprising a substantially folded origami-like structure, or a combination thereof.

17. The system of claim 1, wherein the nanostructure comprises the following:
a. a non-phosphate backbone comprising an amide, a guanidinium, or a triazole linkage;
b. a sugar modified nucleoside or nucleoside analog;
c. a nucleobase modified nucleoside or nucleoside analog; and/or
d. a nucleobase analog.

18. The system of claim 1, wherein the nanostructure comprises the following:
a. a functional group configured for attachment to an electrode; and/or
b. a functional group configured for immobilization of the enzyme.

19. The system of claim 18, wherein the functional group configured for electrode attachment comprises
(a) a thiol on a sugar ring of a nucleoside;
(b) a thiol and a selenol on a nucleobase of a nucleoside;
(c) an aliphatic amine on a nucleoside; and/or
(d) a catechol on a nucleoside;
(e) a RXH or a RXXR, where R is an aliphatic or an aromatic group; and X is a chalcogen preferring to S and Se; and/or
(f) a base chalcogenated nucleoside.
and wherein the functional group configured for immobilization of the enzyme comprises:
(a) an amine functionalized nucleoside that is incorporated into a DNA or a RNA by a chemical or an enzymatic synthesis;
(b) a cyclooctyne and/or a derivative functionalized nucleoside that is incorporated into a DNA and a RNA by a chemical or an enzymatic synthesis; and/or
(c) a catechol functionalized nucleoside that is incorporated into DNA and RNA by chemical or enzymatic synthesis.

20. The system of claim 9, wherein the anchoring molecule comprises one of the following or a combination thereof
(a) a molecule configured to interact with a metal surface through multivalent bonds;
(b) a tripod structure configured to react with a metal surface through trivalent bonds; and
(c) a molecule comprised of a tetraphenylmethane core wherein three of its phenyl rings are functionalized with —CH₂SH or —CH₂SeH and a fourth phenyl ring is functionalized with an azide, a carboxylic acid, a boronic acid, and/or an organic group configured to react with a functional group incorporated into a DNA and/or a RNA nanostructure.

21. The system of claim 9, wherein the anchoring molecule comprises one of the following or a combination thereof:
(a) an N-heterocyclic carbene (NHC);
(b) an N-heterocyclic carbene (NHC) in a metal complex configured to be selectively deposited on a cathode electrode by an electrochemical method in solution;
(c) an N-heterocyclic carbene (NHC) configured to be deposited on both metal electrodes in organic and/or aqueous solutions; and
(d) an N-heterocyclic carbene (NHC) containing functional groups comprising an amine, a carboxylic acid, a thiol, a boronic acid, and/or any organic group configured for attachment.

22. The system of claim 21, wherein
the metal complex comprises Au, Pd, Pt, Cu, Ag, Ti, and/or another transition metal.

23. The system of claim 1, further comprising:
a protein configured to be immobilized at the bottom of the nanogap to support and stabilize the nucleic acid nanostructure.

24. The system of claim 23, wherein
the non-conductive bottom of the nanogap is functionalized with a chemical reagent to immobilize protein, wherein the chemical reagent comprises:
(a) a silane configured to react with an oxide surface;
(b) a silatrane configured to react with an oxide surface;
(c) a multi-arm linker that comprises a silatrane and a functional group;
(d) a four-arm linker that comprises an adamantane core;
(e) a four-arm linker that comprises two silatranes and two biotin moieties; and/or
(f) a four-arm linker that comprises an adamantane core and silatranes and organic functional groups.

25. The system of claim 23, wherein the protein is selected from the group consisting of an antibody, a receptor, an aptamer, and a combination thereof.

26. The system of claim 23, wherein the protein is a streptavidin or an avidin, and the nucleic acid nanostructure is functionalized by a biotin.

27. The system of claim 1, wherein the enzyme is a recombinant DNA polymerase or a recombinant reverse transcriptase that comprises an orthogonal functional group configured to be attached to the nanostructure.

28. The system of claim 27, wherein the recombinant DNA polymerase comprises
(a) an organic group at an N- and/or C-terminal configured for a click reaction on the DNA nanostructure;
(b) an unnatural, modified or synthetic amino acids configured for a click reaction on the DNA nanostructure;
(c) an azide group at an N- and/or C-terminal configured for a click reaction on the DNA nanostructure; and/or (d) a 2-amino-6-azidohexanoic add (6-azido-L-lysine) configured for a click reaction on the DNA and/or RNA nanostructure.

29. The system of claim 28, wherein the nucleic acid nanostructure comprises
(a) a nucleoside with its sugar ring or nucleobase functionalized with an organic group configured for a click reaction; and/or
(b) a nucleoside with its sugar ring or nucleobase functionalized with an acetylene group configured for a click reaction.

30. The system of claim 27, wherein the recombinant reverse transcriptase comprises
(a) an organic group at an N- and/or C-terminal configured for a click reaction on the DNA nanostructure;
(b) an unnatural, modified, or synthetic amino acid configured for a click reaction on the DNA nanostructure;
(c) an azide group at an N- and/or C-terminal configured for a click reaction on the DNA nanostructure; and/or (d) a 2-amino-6-azidohexandic add (6-azido-L-lysine) configured for a click reaction on the DNA and/or RNA nanostructure.

31. The system of claim 1, wherein the biochemical reaction comprises
(a) a reaction catalyzed by a DNA polymerase using DNA as a template and DNA nucleotides as substrates; and/or
(b) a reaction catalyzed by reverse transcriptase using RNA as a template and DNA nucleotides as substrates.

32. The system of claim 31, wherein the DNA nucleotide comprises
(a) a polyphosphate of DNA/RNA nucleosides;
(b) a polyphosphate of DNA/RNA nucleosides tagged with an organic molecule;
(c) a polyphosphate of DNA/RNA nucleosides tagged with an intercalator;
(d) a polyphosphate of DNA/RNA nucleosides tagged with a minor groove binder; and/or
(e) a polyphosphate of DNA/RNA nucleosides tagged with a drug molecule. wherein the polyphosphate comprises two or more phosphate units.

33. The system of claim 32, wherein the polyphosphate is one of the following or a combination thereof:
(a) a hexaphosphate of DNA nucleosides tagged with 1,8-naphthalimide that binds to the DNA nanostructure; or
(b) a hexaphosphate of DNA nucleoside tagged with a derivative of 1,8-naphthalimide that binds to the DNA nanostructure;

34. The system of claim 1 comprises a plurality of nanogaps, each nanogap comprising a pair of electrodes, an enzyme, a nanostructure, and any feature associated with the single nanogap of claim 1.

35. The system of claim 34, wherein the plurality of nanogaps comprises an array of about 100 to about 100 million nanogaps, preferably between about 10,000 to nearly 1 million nanogaps.

36. The system of claim 1, wherein the nucleic acid nanostructure is selected from the group consisting of the DNA nanostructure comprising an origami motif of a Holliday junction (HJ), a multi-arm junction, a double crossover (DX) tile, a triple crossover (TX) tile, a paranemic crossover (PX), a tensegrity triangle, a six-helix bundle, and a single-stranded circular DNA, and a combination thereof, and a DNA tile-like structure with a duplex, a hairpin, a 90°-kink, a kissing-loop, an open 3-way junction, an open 4-way junction, a stacked 3-way junction, or a 3-way loops, and a combination thereof.

37. A method for identifying, characterizing, or sequencing a biopolymer comprising,
(a) providing a non-conductive substrate;
(b) building a nanogap by placing a first electrode and a second electrode next to each other on the substrate;
(c) providing a nanostructure with a sufficient length to bridge the nanogap, wherein the nanostructure comprises a nucleic acid, either a deoxyribonucleic acid (DNA nanostructure) or a ribonucleic acid (RNA nanostructure) or a combination thereof, wherein a base of the nucleic acid is either an unnatural nucleic acid base or the nucleic acid comprises a mixture of unnatural and natural nucleic acid bases;
(d) providing an enzyme that performs a biochemical reaction with the biopolymer;
(e) attaching one end of the nanostructure to the first electrode of the nanogap, and another end to the second electrode wherein the nanogap is bridged, and then attaching the enzyme to the nanostructure; or alternatively, attaching the enzyme to the nanostructure, and then attaching the nanostructure to the nanogap;
(f) providing a bias voltage between the first electrode and the second electrode;
(g) providing a device for recording a current fluctuation through the nanostructure resulting from a distortion within the nanostructure caused by a conformation change initiated by the enzyme attached to the nanostructure; and
(h) providing a data analysis software that is used to characterize or identify the biopolymer or a subunit of the biopolymer.

38. The method of claim 37, wherein the non-conductive substrate is selected from the group consisting of: a silicon, a silicon oxide, a silicon nitride, a glass, a hafnium dioxide, another metal oxide, any non-conductive polymer film, a silicon with a silicon oxide or a silicon nitride or another non-conductive coating, a glass with a silicon nitride coating, another non-conductive organic materials, any non-conductive inorganic materials, and a combination thereof.

39. The method of claim 37, wherein the biopolymer is selected from the group consisting of a DNA, a RNA, an oligonucleotide, a protein, a peptide, a polysaccharide, either natural, modified or synthesized of any of the aforementioned biopolymers, and a combination thereof.

40. The method of claim 37, wherein the enzyme is selected from the group consisting of a DNA polymerase, a RNA polymerase, a DNA helicase, a DNA ligase, a DNA exonuclease, a reverse transcriptase, a RNA primase, a ribosome, a sucrase, a lactase, either native, mutated, expressed, or synthesized of any of the aforementioned enzymes, and a combination thereof.

41. The method of claim 40, wherein the enzyme is selected from the group consisting of T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol ι (iota), Pol κ (kappa), pol η (eta), terminal deoxynucleotidyl transferase, telomerase, either native, mutated, expressed, or synthesized of any of the aforementioned enzymes, and a combination thereof .

42. The method of claim 40, wherein the DNA polymerase is ϕ29 DNA polymerase, either native, mutated, expressed, or synthesized.

43. The method of claim 37,
wherein the two electrodes forming the nanogap are separated by a distance of about 3 nm to about 1000 nm; and
wherein the ends of the electrodes have a substantially rectangular face with a width of about 3 nm to about 1000 nm, and a depth of about 2 nm to about 1000 nm.

44. The method of claim 37,
wherein the two electrodes forming the nanogap are separated by a distance of about 5 nm to about 50 nm; and
wherein the ends of the electrodes have a substantial rectangular face with a width of about 10 nm to about 30 nm, and a depth of about 5 nm to about 20 nm.

45. The method of claim 37, wherein the said electrodes comprise:
e) a metal electrode that can react with a thiol, an amine, a selenol, and another organic functions=al group;
f) a metal electrode that can be functionalized on the surface by a self-assembling monolayer that can react with an anchoring molecule to form a covalent bond;
g) a metal oxide electrode that can be functionalized with silanes that can react with the anchoring molecule to form a covalent bond; or
h) a carbon electrode that can be functionalized with an organic reagent that can react with the anchoring molecule to form a covalent bond.

46. The method of claim 37, futher comprising
passivating the electrodeand the substrate with an insulating layer except for an end surface that faces the nanogap.

47. The method of claim 46, wherein the insulation layer comprises a monolayer or multi-layers of inert chemicals.

48. The method of claim 47, wherein the inert chemical comprises a 11-mercaptoundecyl-hexaethylene glycol (CR-1) fora metal surface passivation, and an aminopropyltriethoxysaline (CR-2) & N-hydroxysuccinimidyl 2-(ω-O-methoxy-hexaethylene glycol)acetate (CR-3) for the substrate surface passivation.

49. The method of claim 37, wherein the unnatural nucleic acid base comprises one of the following or a combination thereof:
(a) a Hachimoji nucleic acid base;
(b) a size expanded nucleobase;
(c) a non-hydrogen bonding nucleobase; and
(d) a universal base.

50. The method of claim 37, wherein the nucleic acid nanostructure comprises one of the following or a combination thereof:
(a) an unpaired or unpairing nucleic acid base; and
(b) an organic superconductor.

51. The method of claim 37, wherein
the nucleic acid nanostructure is self-assembled from either linear or circular DNAs (DNA nanostructure), or linear or circular RNAs (RNA nanostructure), or a combination thereof.

52. The method of claim 37, wherein
the nucleic acid nanostructure has one of the following configurations or a combination thereof:
(d) a substantially one-dimensional geometry, including but not limited to, a linear DNA duplex structure, a linear RNA duplex structure, a linear nucleic acid molecular wire either in helical form or non-helical form, or a combination thereof;
(e) a substantially two-dimensional geometry, including but not limited to, a substantially rectangular structure, a substantially square structure, a substantially triangular structure, a substantially circular structure, or a combination thereof; and
(f) a substantially three-dimensional geometry, including but not limited to, a substantially cylindrical structure, a substantially hollow tube structure, a substantially column-like structure, a geometry comprising a substantially bundle-of-columns structure, a geometry comprising a substantially stacked two-dimensional structure, a geometry comprising a substantially folded origami-like structure, or a combination thereof.

53. The method of claim 37, wherein the nanostructure comprises the following:
a. a non-phosphate backbone comprising an amide, a guanidinium, or a triazole linkage;
b. a sugar modified nucleoside or nucleoside analog;
c. a nucleobase modified nucleoside or nucleoside analog; and/or
d. a nucleobase analog.

54. The method of claim 37, wherein the nanostructure comprises the following:
a. a functional group configured for attachment to electrodes; and/or
b. a functional group configured for immobilization of the enzyme.

55. The method of claim 54, wherein the functional group configured for electrode attachment comprises
(a) a thiol on a sugar ring of a nucleoside;
(b) a thiol and a selenol on a nucleobase of a nucleoside;
(c) an aliphatic amine on a nucleoside; and/or
(d) a catechol on a nucleoside;
(e) a RXH or a RXXR, where R is an aliphatic or an aromatic group; X is a chalcogen preferring to S and Se
(f) a base chalcogenated nucleoside.
and the functional group configured for immobilization of the enzyme comprises:
(a) an amine functionalized nucleoside that is incorporated into DNA and RNA by a chemical or an enzymatic synthesis;
(b) a cyclooctyne and/or a derivative functionalized nucleoside that is incorporated into DNA and RNA by a chemical or an enzymatic synthesis; and/or
(c) a catechol functionalized nucleoside that is incorporated into DNA and RNA by a chemical or an enzymatic synthesis.

56. The method of claim 45, wherein the anchoring molecule comprises one of the following or a combination thereof
(a) a molecule configured to interact with a metal surface through multivalent bonds;
(b) a tripod structure configured to react with a metal surface through trivalent bonds; and
(c) a molecule comprised of a tetraphenylmethane core wherein three of its phenyl rings are functionalized with −CH₂SH and —CH₂SeH and a fourth phenyl ring is functionalized with an azide, a carboxylic acid, a boronic acid, and/or an organic group configured to react with a functional group incorporated into the DNA and/or RNA nanostructure.

57. The method of claim 45, wherein the anchoring molecule comprises one of the following or a combination thereof:
(a) an N-heterocyclic carbene (NHC);
(b) an N-heterocyclic carbene (NHC) in a metal complex configured to be selectively deposited on a cathode electrode by an electrochemical method in solution;
(c) an N-heterocyclic carbene (NHC) configured to be deposited on both metal electrodes in organic and/or aqueous solutions; and
(d) an N-heterocyclic carbene (NHC) containing functional groups comprising an amine, a carboxylic acid, a thiol, a boronic acid, and/or any organic group configured for attachment.

58. The method of claim 57, wherein
the metal complex comprises Au, Pd, Pt, Cu, Ag, Ti, and/or another transition metal.

59. The method of claim 37, further comprising:
providing a protein configured to be immobilized at the bottom of the nanogap to support and stabilize the nucleic acid nanostructure.

60. The method of claim 59, further comprising
functionalizing the non-conductive bottom of the said nanogap with a chemical reagent to immobilize proteins, wherein the chemical reagent comprises:
(g) a silane configured to react with an oxide surface;
(h) a silatrane configured to react with an oxide surface;
(i) a multi-arm linker that comprises a silatrane and a functional group;
(j) a four-arm linker that comprises an adamantane core;
(k) a four-arm linker that comprises two silatranes and two biotin moieties; and/or
(l) a four-arm linker that comprises an adamantane core and silatranes and organic functional groups.

61. The method of claim 59, wherein the protein is selected from the group consisting of an antibody, a receptor, an aptamer, and a combination thereof.

62. The method of claim 59, wherein the protein is a streptavidin or an avidin, and the nucleic acid nanostructure is functionalized by a biotin.

63. The method of claim 37, wherein the enzyme is a recombinant DNA polymerase or a recombinant reverse transcriptase that has an orthogonal functional group configured to be attached to the nanostructure.

64. The method of claim 63, wherein the recombinant DNA polymerase comprises
(a) an organic group at an N- and/or C-terminal configured for a click reaction on the DNA nanostructure;
(b) an unnatural, modified or synthetic amino acids configured for a click reaction on the DNA nanostructure;
(c) an azide group at an N- and/or C-terminal configured for a click reaction on the DNA nanostructure; and/or
(d) a 2-amino-6-azidohexanoic add (6-azido-L-lysine) configured for a click reaction on the DNA and/or RNA nanostructure.

65. The method of claim 64, wherein the nucleic acid nanostructure comprises
(a) a nucleoside with its sugar ring or nucleobase functionalized with an organic group configured for a click reaction; and/or
(b) a nucleoside with its sugar ring or nucleobase functionalized with an acetylene group configured for a click reaction.

66. The method of claim 63, wherein the recombinant reverse transcriptase comprises
(a) an organic group at an N- and/or C-terminal configured for a click reaction on the DNA nanostructure;
(b) an unnatural, modified, or synthetic amino acid configured for a click reaction on the DNA nanostructure;
(c) an azide group at an N- and/or C-terminal configured for a click reaction on the DNA nanostructure; and/or
(d) a 2-amino-6-azidohexanoic add (6-azido-L-lysine) configured for a click reaction on the DNA and/or RNA nanostructure.

67. The method of claim 37, wherein the biochemical reaction comprises
(a) a reaction catalyzed by a DNA polymerase using DNA as a template and a DNA nucleotide as a substrate; and/or
(b) a reaction catalyzed by reverse transcriptase using RNA as a template and a DNA nucleotide as a substrate.

68. The method of claim 67, wherein the DNA nucleotide comprises
(a) a polyphosphate of DNA/RNA nucleoside;
(b) a polyphosphate of DNA/RNA nucleoside tagged with an organic molecule;
(c) a polyphosphate of DNA/RNA nucleoside tagged with an intercalator;
(d) a polyphosphate of DNA/RNA nucleoside tagged with a minor groove binder; and/or
(e) a polyphosphate of DNA/RNA nucleoside tagged with a drug molecule. wherein the polyphosphate comprises two or more phosphate units.

69. The method of claim 68, wherein the polyphosphate is one of the following or a combination thereof:
(c) a hexaphosphate of DNA nucleoside tagged with 1,8-naphthalimide that binds to the DNA nanostructure; and
(d) a hexaphosphate of DNA nucleoside tagged with a derivative of 1,8-naphthalimide that binds to the DNA nanostructure;

70. The method of claim 37, wherein the nucleic acid nanostructure is selected from the group consisting of the DNA nanostructure comprising an origami motif of a Holliday junction (HJ), a multi-arm junction, a double crossover (DX) tile, a triple crossover (TX) tile, a paranemic crossover (PX), a tensegrity triangle, a six-helix bundle, and a single-stranded circular DNA, or a combination thereof, and a DNA tile-like structure with a duplex, a hairpin, a 90°-kink, a kissing-loop, an open 3-way junction, an open 4-way junction, a stacked 3-way junction, or a 3-way loops, or a combination thereof.
